(19) United States
(12) Patent Application Publication
(10) Pub. No.: US 2014/0037570 A1
(43) Pub. Date: Feb. 6, 2014

(54) NBPT solution for preparing urease inhibited urea fertilizers prepared from N-alkyl; N, N-alkyl; and N-alkyl-N-alkoxy amino alcohols

(71) Applicants: Garnett B. Whitehurst, New Bern, NC (US); Brooks M. Whitehurst, New Bern, NC (US)

(72) Inventors: Garnett B. Whitehurst, New Bern, NC (US); Brooks M. Whitehurst, New Bern, NC (US)

(73) Assignee: Whitehurst Associates Inc.

(21) Appl. No.: 13/507,848

(22) Filed: Aug. 2, 2012

(51) Int. Cl. C09K 15/22; A61L 11/00; C05C 9/00

(52) U.S. Cl. 424/76.6; 252/403; 71/30

(57) ABSTRACT

Solutions are prepared by dissolving N-(n-butyl)-thiophosphoric triamide (NBPT) in one or more N-alkyl amino alcohols, N,N-dialkyl amino alcohols, N-alkyl-N-alkoxy amino alcohols, and mixtures thereof. The solutions may be used in urea fertilizers to reduce nitrogen volatilization, or to reduce the odor of animal waste. Methods of preparing urea fertilizers and the resultant products are also described.

### BACKGROUND OF THE INVENTION

(1) Field of the Invention

The present invention relates to solutions for use in reducing nitrogen volatilization comprising N-(n-butyl)-thiophosphoric triamide (NBPT) dissolved in one or more N-alkyl amino alcohols, N,N-dialkyl amino alcohols, N-alkyl-N-alkoxy amino alcohols, and mixtures thereof, to methods of making fertilizers using these solutions, and to the resultant fertilizers. The solutions also find utility in reduction of odors from animal wastes, and other materials containing urine.

(2) Description of the Prior Art

Urea is a commonly used nitrogen source in agriculture which is subject to degradation in the soil by action of the enzyme urease. This degradation leads to loss of nitrogen as ammonia in a process known as volatilization. A number of approaches have been tried to protect urea from volatile nitrogen loses including the use of metal inhibitors such as copper salts, or zinc salts, boric acid salts (borates), or sulfur coatings; however; the most effective method is the use of an organic urease inhibitor.

N-(n-butyl)-thiopsosphoric triamide (NBPT) is a known urease inhibitor described by (Kolc et. al. U.S. Pat. No. 4,530,714). The compound is a waxy solid with poor water solubility making it difficult to coat urea and achieve adhesion of the compound. The compound undergoes hydrolysis and is thermally unstable.

In the soil, NBPT converts to the phosphoric triamide form (oxon analog) which is the more potent but much more unstable inhibitor (McCarthy, G. W., Bremner, J. M., and Chai, H. S. “Effect of N-(n-butyl)-thiophosphoric triamide on the hydrolysis of urea by plant, microbial and soil urease.” Biology and Fertility of Soils Volume 8 Pages 123-127, 1989). For commercial use it is desirable to protect the sulfur atom of the thiophosphoric triamide structure of NBPT until it reaches the soil.

The 714 patent describes the mixing of NBPT with organic solvents (acetone, disobutylketone, methanol, ethanol, 2-propanol, ether (diethyl), toluene, methylene chloride) to distribute the compound into the soil in an effective concentration range which can be anywhere from 5 ppm to 100 ppm depending upon the soil. The organic solvents described by the 714 patent are either too flammable for use or pose significant health risks to be considered suitable for coating urea granules.

In an alternate method, the 714 patent indicates that NBPT can be mixed with solids such as gypsum or clay to distribute the compound into the soil in an effective concentration.

Omilinsky et. al. (U.S. Pat. No. 5,698,003) describes the dissolution of NBPT with a glycol such as propylene glycol or ethylene glycol and esters of glycols. Glycols are compounds with adjacent alcohol groups in the chemical structure. The glycol solvent may contain a co-solvent liquid amide such as N-methyl-2-pyrrolidine and potentially a surfactant or dispersing agent such as polyethylene glycol or esters of polyethylene glycol (polyether alcohols). The patent indicates that esters of glycerol (a triol) may be used as the base solvent. Urea granules containing NBPT are prepared by mixing the urea granules with the NBPT dissolution solvent. Omilinsky et. al. teach that a drying agent such as clay or gypsum may be added to the compositions in the event that a product with excessive wetness is obtained.

Weston et al. (U.S. Pat. No. 5,352,265 and U.S. Pat. No. 5,364,438) teach the dissolution of NBPT in liquid amides such as 2-pyrrolidone or N-alkyl-2-pyrrolidones such as N-methyl-2-pyrrolidone to prepare both solid urea formulations (265 patent) or liquid formulations (438 patent).

Hojjatie et al. (US 2006/0185411) teach the use of a number of sulfur salts of calcium or magnesium (calcium polysulfide, thiosulfate, and magnesium thiosulfate) as urease inhibitors to prepare granular or liquid urea compositions.

Quin (US 2004/0163434) teaches the formation of sulfur coated urea which may contain the urease inhibitor NBPT supplied from a proprietary liquid formulation sold as Agrotain® and distributed by Koch Agronomic Services, Kansas, USA.

Sutton et al. (U.S. Pat. No. 5,0247,689) teach the formation of a liquid fertilizer that includes urease inhibitors such as NBPT and nitrification inhibitors such as dicyandiamide in aqueous mixtures of urea ammonium polyphosphate, ammonium thiosulfate and potentially other plant growth improving compounds.

Sutton (US 2007/0295047) teaches the formation of a solid fertilizer comprised of urea and a urea-formaldehyde polymer which may additionally include a urease inhibitor such as NBPT.

Sutton et al. (US 2007/0157689) describes a fertilizer additive composed of urea, a urea-formaldehyde polymer and NBPT dissolved in an N-alkyl-2-pyrrolidone.

Hamad et al. (US 2007/0077428) suggests the formation of odor inhibiting fibers (diapers) comprised of a cellulosic fiber and an odor-inhibiting formulation. The odor inhibiting formulation is comprised of an odor inhibiting agent dissolved in hydrophilic or hydrophobic solvent and mixtures of hydrophobic and hydrophilic solvents. The hydrophilic solvents could include amino alcohols such as ethanolamine and diethanolamine. Acids may be added to the odor-inhibiting fiber formulations to neutralize ammonia which may be formed by breakdown of urea. Hamad et. al. (7428 patent) suggests that urease inhibitors such as NBPT may be included in the odor-inhibiting formulation.

Sun et al. (U.S. Pat. No. 6,852,904) suggests the formation of odor controlling cellulosic fibers such as diapers or medical absorbent garments in which a carboxylic acid or partially neutralized carboxylic acid are employed to form the odor-inhibited cellulosic product. The odor-inhibiting formulation may include a transition metal as a hydroxide or oxide which may be used to partially neutralize the carboxylic acid groups.

Cigler (WO 2008/000196) teaches the formation of a solvent system for thiophosphoric triamide solutions comprised of one or more glycol ethers which may optionally contain substances to improve the stability of the thiophosphoric triamde. Examples of stabilizing agents include polyvinylpyyrolidone, N-methylpyrrolidone as crystallization inhibitors. Examples of glycolethers suited to the teachings are diethyleneglycolmonomethylether, dipropyleneglycolether, monomethylether and triethyleneglycolmonomethylether.

Whitehurst et al. (U.S. Pat. No. 8,163,058) teach the formation of fertilizer materials such as granular urea or liquid urea formulations in which the urea is treated with a solution containing NBPT which has been dissolved in an amino alcohol such as diethanolamine, triethanolamine, diisopropanolamine, etc. All carbon chains attached to the nitrogen atom in the solvent system described Whitehurst et. al. contain an alcohol group (058 patent).

Whitehurst et al. (U.S. Pat. No. 8,048,189) teach the formation of a solution of NBPT in a buffered mixture composed of the reaction product of an amino alcohol with a carboxylic acid up to 6 carbons in length.

Whitehurst et al. (U.S. Pat. No. 8,133,294) teaches the formation of various urea containing fertilizers from the buffered mixture of the reaction product of an amino alcohol and a carboxylic acid up to 6 carbons in length. Whitehurst et. al. (U.S. Pat. No. 8,048,189) note that the stability of NBPT is affected by pH when water is present and it is undesirable to formulate a mixture with a pH below 7. All carbon chains attached to the nitrogen atom in the contain an alcohol group in the amino alcohols used to prepare buffered solvents for NBPT in the teachings of Whitehurst et al. (189 and 294 patents).

Urea is a high nitrogen analysis material which is often desirable as a starting material for making additional fertilizer products providing phosphorus or potassium as primary nutrients, calcium, magnesium or sulfur as secondary nutrients or micronutrients such as boron, copper, iron, manganese, molybdenum and zinc.

Whitehurst et al. (U.S. Pat. No. 6,030,659) teaches the formation of phosphate coated urea by first reacting urea with an acid then adding an apatite mineral phosphate source to the surface. Reaction of phosphoric acid while on the urea surface with the apatite mineral is expected to solubilize the apatite mineral to provide available phosphate. It is expected that the acidified coating would help to reduce volatile nitrogen losses from urea.

Whitehurst et al. (U.S. Pat. No. 6,830,603) describes a coating methodology wherein boron containing urease inhibitor compositions may be used to add additional nutrients such as phosphate, potassium, etc. The coating of urea with other materials is known and the references in Whitehurst et al. (603 patent) provides a partial summary of prior art in the area. The inhibitors and binders taught in the 603 patent are aqueous mixtures that include ethanolamine borates, diethanolamine borates or triethanolamine borates and mixtures of these.

Urea is a common component of animal wastes (manures, green manures, animal bedding materials contaminated with urea, etc.). These animal wastes release ammonia as they decompose due to the action of the enzyme urease. Kolc et. al. (U.S. Pat. No. 4,517,003) disclosed the use of N-acylphosphoric triamides as urease and/or nitrification inhibitors. Kolc et al. (003 patent) include manures in the fertilizers which may be protected from volatile ammonia loss by using a phosphoric triamide. The urease/nitrification inhibitor may be distributed in either liquid form (dissolved in alcohols or halogenated solvents) or in solid form (mixed with clays, vermiculite, gypsum e.g.) to distribute the phosphoric triamide onto a fertilizer (003 patent).

Weissemeier et al. (U.S. Pat. No. 8,075,659) describe the use of urease inhibited fertilizer formulations in which two phosphoric triamide urease inhibitors are used. The phosphoric triamides may be present in the thiophosphoric triamide form. Liquid manures may be treated with the combination of urease inhibitors.

Sheets (U.S. Pat. No. 7,422,680) teach the use of a urease inhibitor such as NBPT in the pretreatment of animal wastes to prevent ammonia release when making fertilizers from animal wastes.

Aylen et al. (U.S. Pat. No. 7,434,540) teach the use of clay based animal bedding material as an absorbent to reduce ammonia levels, odor, microorganisms and insects in animal stalls. The clay based absorbent may contain NBPT as a urease inhibitor.

The use of NBPT or other phosphoric and/or thiophosphoric triamides requires a dispersal mechanism and most of the patents previously identified deal with materials which can be used to disperse NBPT, other thiophosphoric triamides, and phosphoric triamides in liquids or solids. The teachings of the prior art indicate that liquid dispersal agents (solvents) are desired which are safe to handle, easily mix with water, that are stable under somewhat broad temperature conditions and protect the compounds from degradation to unstable forms that are less active as urease inhibitors. A desirable feature for the solvent systems for NBPT is their ability to keep the NBPT dissolved under low temperature conditions. This is made somewhat difficult by the endothermic heat of solution of NBPT and therefore its tendency to crystallize from concentrated solutions at low temperature.

Amino alcohols are prepared commercially by adding ammonia to ethylene oxide (oxirane), propylene oxide(methyloxirane) where the cyclic oxygen containing ring is opened by ammonia and an alcohol functional group is created at one carbon atom of the chain and an amino group is added to an adjacent carbon atom of the chain. The amino group can react with another molecule of ethylene oxide to form diethanolamine and that amino alcohol can react with another molecule of ethylene oxide to form triethanolamine. The mixture of compounds is separated to obtain the desired product or sometimes sold as a mixture.

The reaction with ethylene oxide may be conducted using an amine (e.g., methylamine, dimethylamine, ethylamine, etc) in place of ammonia which results in the formation an N-alkyl or an N,N-dialkyl amino alcohols and possibly N-alkyl-N-alkoxy amino alcohols. When the starting material is ethylene oxide (oxirane) the products from the reaction will be N-alkyl, N,N-dialkyl; or N-alkyl-N-alkoxy substitutions of either ethanolamine or diethanolamine. The reaction mixture can be separated if desired to obtain the pure materials needed. In some cases, the mixture itself may have desirable properties or be difficult to further purify and in those situations the mixture might be sold without greater purification.

The reaction of propylene oxide(methyloxirane) with an amine would generate N-alkyl, N,N-alkyl and N-alkyl-N-alkoxy substituted propanolamines or N-alkyl, N,N-alkyl and N-alkyl-N-alkoxy substituted isopropanolamines. The mixture of products can be separated or sold as a mixture when desired. The 4 carbon group of amino alcohols containing N-alkyl, N,N-alkyl and N-alkyl-N-alkoxy substitutions can be generated by reaction of an amine with 2,3-dimethyloxirane, 2,2-dimethyloxirane and 2-ethyloxirane.

Some amino alcohols tend to freeze easily at temperatures which could be encountered in industrial practice. As an example, triethanolamine (TEA) freezes at 20.5° C., diethanolamine (DEA) freezes at 28° C., and consequently heated storage is often required for high purity TEA or DEA. A technique often used to prevent the freezing of the material is to add water and reduce the TEA or DEA concentration to 85%. However, this reduces the stability to crystallization of NBPT solutions in TEA and/or DEA. Thus, it is desirable to have a mechanism which can prevent the crystallization of NBPT in solutions with TEA and/or DEA that does not involve adding water.

Because TEA and DEA tend to be viscous liquids they are desirable as solvents for forming some powder coated granular products. The high viscosity allows adhesive forces to form which may help with stabilizing the powder coating on a granular substrate. At low temperature the high viscosity makes the binding solution difficult to handle since viscosity increases in a manner inversely related to temperature. Consequently, solutions of NBPT in either TEA and/or DEA can become difficult to use at low temperature due to their viscosity. Thus, it is desirable to have a method which will reduce the viscosity of the NBPT solution of TEA and/or DEA which reduces the crystallization tendency of NBPT and allows the solution to flow at low temperature.

### SUMMARY OF THE INVENTION

In accordance with the present invention, solutions for use in reducing nitrogen volatilization are prepared by dissolving N-(n-butyl)-thiophosphoric triamide (NBPT) in one or more N-alkyl amino alcohols, N,N-dialkyl amino alcohols, N-alkyl-N-alkoxy amino alcohols, and mixtures thereof. The solutions may optionally contain one or more carboxylic acids having up to 24 carbon atoms to reduce nitrogen volatilization.

The solutions may be applied as a coating for granular urea fertilizers, or mixed with aqueous urea fertilizer solutions. The urea fertilizers may optionally contain other materials such as plant nutrients including micronutrients and denitrification inhibitors. The NBPT containing solutions of N-alkyl, N,N-dialkyl and/or N-alkyl-N-alkoxy amino alcohols may be prepared with other amino alcohols (not substituted by alkyl groups) to control the crystallization points of the NBPT solutions in amino alcohols. The NBPT containing solutions, also, may be used to treat animal wastes containing urea to prevent decomposition of the urea present. Denitrification inhibitors may be included when treating animal wastes.

The term alkyl substituted amino alcohol (abbreviated—ASAA) will be used to refer to compounds which are members of the N-alkyl or N,N-dialkyl amino alcohols or N-alkyl-N-alkoxy amino alcohols as defined below.

The term alkanolamine will be used to include compounds such as ethanolamine, diethanolamine, triethanolamine, propanolamine, isopropanolamine, dipropanolamine, diisopropanolamine, tripropanolamine, trisopropanolamine and the 4 carbon butanolamine family which includes numerous branched structures.

In general the solvents for N-(n-butyl)thiophosphoric triamide (NBPT) needed to practice the invention are liquids which could be broadly classified as alkyl substituted amino alcohols (ASAA). These alkyl substituted amino alcohols can be further described as N-alkyl or N,N-dialkyl amino alcohols of the general formula 1:

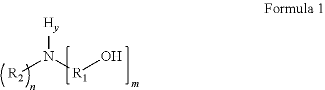

where R₁ is a carbon chain from 2 to 4 carbon atoms and R₂ is a carbon chain with 1 to 4 carbon atoms; m, n, are integers with values of 1 or 2; y is an integer with values of 0 or 1 such that m+n+y=3; and when y=0 then m+n=3. R₁ and/or R₂ may be branched structures. It is to be further understood that R₂ will not contain hydroxyl (alcohol groups).

Another group of compounds usable as solvents for NBPT are related to the compounds above but contain an ether linkage in a carbon chain attached to nitrogen and may be described as N-alkyl-N-alkoxy amino alcohols of formula 2:

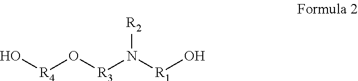

where R₁, R₃ and R₄, are a carbon chains with from 2 to 4 carbon atoms and R₂ may be a carbon chain of 1 to 4 carbon atoms. All carbon chains could be branched and it is further understood that when the N-alkyl-N-alkoxy amino alcohol was formed by reacting an oxirane with an amine that R₁, R₃ and R₄ will have the same structure. It is further understood that R₂ will not contain hydroxyl (alcohol) groups.

A mixture N-alkyl, N,N-dialkyl and N-alkyl-N-alkoxy amino alcohols or a mixture of compounds of formula 1 and formula 2 could be used to prepare the solvent system for NBPT. The mixtures of N-alkyl, N,N-dialkyl and N-alkyl-N-alkoxy amino alcohols would be derived from the reaction of an amine with ethylene oxide (oxirane), propylene oxide(methyloxirane), 2,2-dimethyloxirane, 2,3-dimethyloxirane, or 2-ethyloxirane. Reactions of oxiranes with amines produce mixtures which may be separated or used as produced. Alternatively, the mixture of N-alkyl, N,N-dialkyl and N-alkyl-N-alkoxy amino alcohols could be formed by mixing purified compounds within any of the three groups of N-alkyl, N, N-dialkyl or N-alkyl-N-alkoxy amino alcohols.

The factors which should be considered when selecting the ASAA or mixture of ASAA to use as a solvent for NBPT include: solubility limit for NBPT, ability of NBPT solution derived from ASAA to withstand low temperature by not forming crystals of NBPT, flammability, and solubility of the resulting solutions in water. NBPT has an endothermic heat of solution and solutions containing the compound may form crystals at low temperature. These crystals of NBPT increase the handling difficulty at low temperature. Materials that can inhibit crystal formation and increase the low temperature usefulness of NBPT solutions are particularly important where nitrogen solutions or granular urea may be used when the ground is still cold and it is necessary to treat urea to protect it from degradation by urease.

The N-alkyl substituted amino alcohols which could be used to practice the invention include: N-methylethanolamine (NMEA), N-ethylethanolamine, N-propylethanolamine, N-isopropylethanolamine, N-butylethanolamine, N-sec-butylethanolamine, N-isobutylethanolamine and N-tert-butylethanolamine, N-methyldiethanolamine (MDEA), N-ethyldiethanolamine, N-propyldiethanolamine, N-isopropyldiethanolamine, N-butyldiethanolamine, N-sec-butyldiethanolamine, N-isobutyldiethanolamine, N-tert-butyldiethanolamine. Similar alkyl substituted propanolamines, isopropanolamines, dipropanolamines, diisopropanolamines or butanolamines including the branched butanolamine structures could be used in the practice of the invention.

The N,N-dialkyl substituted amino alcohols which could be used to practice the invention include: N,N-dimethylethanolamine (DMEA), N,N-diethylethanolamine (DEEA), N,N-dipropylethanolamine, N,N-diisopropylethanolamine, N,N-dibutylethanolamine, N,N-disecbutylethanolamine, N,N-diisobuytlethanolamine and N—N-tert-butylethanolamine. Similar N,N-dialkyl substituted propanolamines, isopropanolamines or butanolamines including the branched butanolamine structures could be used in the practice of the invention. Additionally, the N,N-alkyl substitutions on the amino alcohol need not be identical, e.g. N-ethyl-N-methylethanolamine and similar substitutions are understood to be included in N,N-alkyl substituted amino alcohols needed to practice the invention.

The N-alkyl-N-alkoxy amino alcohols which could be used to practice the invention include: 2-((2(2-hydroxyethoxy)ethyl)(methyl)amino)ethanol (MHEEA), 2-(ethyl(2-(2-hydroxyethoxy)ethyl)amino)ethanol, 2-((2-(2-hydroxyethoxy)ethyl)(propyl)amino)ethanol, 3-(ethyl(3-(3-hydroxypropoxy)propyl)amino)propan-1-ol and 3-((3-hydroxypropoxy)propyl)(methyl)amino)propan-1-ol.

Additionally, reaction mixtures from the production of N-alkyl amino alcohols that contain N-alkyl-N-alkoxy amino alcohols are particularly useful as solvents for NBPT. One such example is a mixture of N-methyldiethanolamine (MDEA) and 2-((2(2-hydroxyethoxy)ethyl)(methyl)amino)ethanol (MHEEA) which is commercially available and sold under the name of Amine G2 by Dow Chemical Company Michigan, USA. The product is sold with a varying content of MDEA and MHEEA which may include other compounds. The amine G2 mixture may have a ratio of MHEEA to MDEA of about 0.5:1 to 99:1, although a ratio of MHEEA to MDEA of 10:1 to 25:1 is more typical.

To form a solution of N-(n-butyl)thiophosphoric triamide (NBPT) dissolved in an alkyl substituted amino alcohol (ASAA) or mixture of ASAA, NBPT is melted into liquefied ASAA or mixture to dissolve the NBPT. The temperature should be maintained below 50° C. while dissolving the NBPT to form the initial solution. Once formed the NBPT solution in an ASAA or mixture of ASAA may be used to treat aqueous urea mixtures to prevent formation of ammonia from urea, to treat granular urea and to form nutrient powder coated urea products with NBPT which have reduced tendency to form ammonia, and to treat animal wastes to reduce the evolution of ammonia from urea. Optionally the pH of the solution may be adjusted as indicated below to prepare products when the starting materials may release ammonia naturally.

The NBPT concentration of the final solution thus formed could range from about 0.5% NBPT to about 40% NBPT. The upper bound is determined by the solubility limit of NBPT in the solution and is affected by the temperature range where the product will be stored and handled. The lower bound is determined by the application where the solution might be used. Some applications identified below for the NBPT containing solutions of the invention require low concentrations of NBPT and will be less affected by storage conditions.

In an alternate method of producing NBPT containing solutions, the alkyl substituted amino alcohols (ASAA) or mixtures of ASAA may be used to inhibit crystallization of a solution of NBPT in alkanolamines. The crystallization can be inhibited by adding an ASAA or mixtures of ASAA and the alkanolamine together when forming an NBPT containing solution in the alkanolamine. A solution is formed by mixing the alkanolamine or mixture of alkanolamines with an ASAA or mixture of ASAA in any order and then forming the NBPT containing mixture as indicated previously. The solution has the same uses as those identified for the NBPT solution in an ASAA or a mixture of ASAA.

In general NBPT solutions prepared from the ASAA or mixtures of ASAA including the crystallization inhibited solutions of NBPT formed by mixing ASAA with using alkanolamines or a mixture of alkanolamines will have a high pH. This can cause ammonia to release from an aqueous solution if ammonia or ammonium salts are present. The high pH will additionally cause ammonia to be released from some granular materials.

En situations where pH control is needed to prevent degradation of a granular or solid material the pH of the NBPT solution in the ASAA may be adjusted with a carboxylic acid thus, producing a mixture of the ASAA ammonium ions; ASAA; and carboxylate ions of the carboxylic acid. The mixture will additionally contain water which is generated by the reaction of the carboxylic acid and the ASAA. The exact mixture of ammonium ions of the ASAA and the ASAA will depend upon the molar ratio of the carboxylic acid to the ASAA, the ionization constant of the ASAA, the pH of the NBPT solution in the ASAA provided it above the pKₐ of the carboxylic acid. Should the pH be below the pKₐ of the carboxylic acid then the solution will contain the ammonium ion of the ASAA, the carboxylate ion, and the carboxylic acid which did not ionize in the reaction with the ASAA. The pH adjusted solution of NBPT in an ASAA is then used to prepare granular or liquid urea fertilizers, or treat manures.

The acids needed to practice the invention may be described as carboxylic acids with from 2 to about 24 carbons provided that the acid exists as a liquid at room temperature or which may liquefy when heated and held in the heated state at about 40° C. Examples carboxylic acids which are items of commerce include: acetic, propionic, butyric, valeric, caproic, oleic, linoleic etc. The acids may additionally have an alcohol group such as lactic acid (2-hydroxypropionic acid) which is an item of commerce.

Alternatively, the pH adjusted solution of NBPT may be prepared from a mixture of ASAA and alkanolamines or mixture of alkanolamines which was formed to control the crystallization temperature and viscosity of the pH adjusted solution containing NBPT in an alkanolamine or mixture of alkanolamines. When preparing the pH adjusted NBPT solutions with ASAA of mixtures of ASAA and alkanolamines or mixtures of alkanolamines the order of addition of the ASAA or mixture of ASAA and alkanolamine or mixture of alkanolamines is not critical. A mixture of pH adjusted solution of NBPT in ASAA or alkanolamines may be mixed to achieve the final product solution. These pH adjusted NBPT containing mixtures of ASAA and alkanolamines which have reduced viscosity and reduced tendency towards NBPT crystallization may be used to treat granular urea, aqueous urea solutions, form coated granular urea products with additional nutrients, or treat animal wastes.

All of the previously described solutions of NBPT may be used to prepare granular urea fertilizers. These products are preferably prepared from the pH adjusted solutions to reduce the tendency for ammonia release from the urea granule wetted by the solution of the present invention. The NBPT treated granular urea is formed by simply adding the NBPT containing solution to granular urea and mixing to distribute the liquid. Any commercially available mixing equipment can be used to treat the granular urea product with one of the NBPT containing solutions of the invention. The resultant fertilizer product is comprised of urea granules coated with NBPT dispersed in the solvent.

All of the previously described solutions of NBPT may be used to treat aqueous urea solutions. The solutions without pH adjustment could be used when the solution already has ammonia present.

All of the previously described solutions of NBPT may be used to treat animal waste materials. Preferably, the pH adjusted NBPT containing mixtures in ASAA would be used for the purpose of treatment of an animal waste material. The NBPT solution in an ASAA solution would be used to reduce the odor of the animal waste material.

All of the previously described solutions of NBPT can be used to prepare aqueous urea solutions which contain additional nutrients and NBPT. The additional nutrients can be added as liquids or solids.

All of the previously described solutions can be used to form granular urea products with additional nutrients that contain NBPT. The additional nutrients are typically added to the surface of the granular urea in powdered form. The powders can be produced by any dry grinding process. The production of granular urea with additional nutrients containing NBPT may require the use of additional liquid acting as a binding agent or as a liquid which can serve as both a diluent for the NBPT containing solution and binding agent. The resultant fertilizer is comprised of urea granules, with a first coating of NBPT dispersed in the selected solvent, components of an optional diluent for the NBPT solvent or components of an additional liquid binding agent, and an outer coating of the additional nutrients. The outer coating could be applied in several layers.

The amount of urease inhibitor NBPT needed in a given urea fertilizer product produced according to the invention often depends upon the soil type and soil pH and the amount of urease activity due to soil bacteria. The quantity of urease inhibitor needed in the final liquid or granular product treated with an NBPT containing solution could be determined by measuring the urease activity in a range of soils and then determining the amount of inhibitor needed to inhibit that amount of urease in the specific soil where the urea containing products will be applied. The alternative method involves assessing the volatile nitrogen losses from a range of soils and formulating with the needed NBPT containing solution to achieve control of the volatile nitrogen loss in the specific soil where the liquid or granular treated products will be applied.

Generally the effective amount of NBPT which will need to be added to treat urea fertilizers will lie within the range of 0.005% to 0.25% depending upon the product and the conditions where the NBPT containing product will be applied. When treating an animal waste, dilution of a concentrated NBPT solution with an aqueous solution may be required to achieve an effective dose within the range of 0.005% to 0.25%.

As used herein, the term “urea fertilizer” encompasses urea and mixtures of urea with other primary nutrients, secondary nutrients and/or micronutrients. Preferably, urea comprises at least 10% by weight of the urea fertilizer (dry weight basis). Unless otherwise noted all percentages refer to weight percentages or parts per 100 parts.

As used herein the term animal waste is understood to include manures, green manures, animal bedding materials or other products which could contain urea derived from the liquid and/or solid excrement of any animal. These animal waste products are sometimes called reduced nitrogen fertilizers.

The term solubility limit as used below refers to the measurement of maximum amount of NBPT which will dissolve in an ASAA, mixture of ASAA, crystallization inhibited mixtures of an alkanolamine or mixture of alkanolamines with ASAA or mixtures of ASAA. The term solubility limit would also apply to the pH adjusted solutions of NBPT dissolved in an ASAA, mixture of ASAA, crystallization inhibited mixtures of an alkanolamine or mixture of alkanolamines with ASAA or mixtures of ASAA. The NBPT containing solution is observed over a period of time to assess whether solids form upon standing. If solids form upon standing the solution is deemed unstable and the solubility limit is considered exceeded. All solubility limits are expressed in weight percentages and are understood to imply the limit of the solubility of NBPT.

Additionally, the practice of the invention may include other items of commerce including ethylene glycol and propylene glycol. The glycols can be used as co-solvents to control spreadability of a formulation. Additionally, other materials such as glycol ethers and/or nonionic surfactants may be included in the formulation to improve sprayability and/or surface coating behavior. The glycol ethers and/or nonionic surfactants quite often work by lowering the surface tension of aqueous solutions to permit the liquid to more effectively wet a surface. The glycol ethers may increase the adhesion of the liquid coating to the fertilizer substrate.

Additionally a colorant or mixture of colorants may be used to help the user determine when the urea containing material has been treated. In some cases it may be necessary to add some alcohol to the solution of the present invention containing NBPT to help disperse the colorant(s).

The production of some coated granular products may include one or more sources of additional plant nutrients as water soluble salts such as ammonium sulfate, monoammonium phosphate, potassium chloride, potassium dihydrogen phosphate, potassium sulfate, and salts of iron, copper, zinc, manganese, and others; and partially water soluble salts such as gypsum, potassium magnesium sulfate and others commonly employed in agricultural practice. The only requirement for the selection of the additional plant nutrient source is that of compatibility with urea. Compatibility of many fertilizer materials can be determined from the “Farm Chemicals Handbook” published by Meister Publishing Co. Ohio, USA.

To make coated granular urea fertilizers containing additional nutrients using the NBPT containing solution of the invention, one or more materials providing plant nutrients other than urea is preferably used in a powdered form. The term powder for purposes of the invention shall mean any finely divided substance prepared by some dry grinding process. There are numerous forms of dry grinding equipment available including hammer mills or pin mills, etc. A powder for purposes of the invention shall imply any finely divided material with a particle size less than 0.300 mm (300 μm).

When preparing the coated granular urea fertilizers with powdered nutrients, the NBPT containing solution will generally need to be diluted to concentrations below about 5% to avoid using too much NBPT in the product. The dilution may be made in a number of aqueous liquids, liquids such an aqueous ethanolamine borate, diethanolamine borate, triethanolamine borate sold under the trade name of Arborite® and available from EnCee Chemical, PO Box 39|New Bern, N.C. 28563, USA (Telephone: 252-633-5868). The diluent liquid could be an available aqueous liquid fertilizer solution. Alternatively, the NBPT containing solutions of the present invention may be prepared at a lower concentration to prepare the powder coated product. An NBPT containing solution of the present invention may be mixed with another liquid such as aqueous urea solution to provide the liquid required to cause adhesion of the powder(s) to granular urea. To enhance the properties of the diluent liquid, denitrification inhibitors may be added to the diluent solution to produce coated urea products with additional nutrients which are protected from both volatile nitrogen losses and nitrification losses.

To form the powder coated granular urea product, the granular urea may be treated with the NBPT containing solution and other binding agents or nitrification enhanced diluent to form a surface wetted urea product and then the powdered nutrients are added to complete the coated NBPT containing granular product. In an alternate method suited to continuous processing, the powder may be mixed with the granular urea and then an NBPT containing solution of the present invention or a diluted NBPT formulation of the present invention added while continuing to mix the combined mass until the powder has adhered to the urea. In a another alternative method of forming the powder coated urea product containing NBPT, granular urea previously treated with the NBPT containing solutions of the present invention may be used and then another binding agent employed to cause the powders to adhere to the urea employed. Any commercially available mixing equipment may be employed to prepare the powder coated urea product containing NBPT and additional plant nutrients,

Fertilizer compositions are described by expressing the weight percentage of the primary elements present in the following manner: XX-YY-ZZ; where XX is the percentage of nitrogen, YY is the phosphate percentage expressed as P₂O₅, and ZZ is the potassium content expressed as the percentage K₂O. When secondary elements are present the percentages are often listed after the primary elements in the order calcium, magnesium and sulfur or by stating the analysis for the secondary element followed by the symbol for the element. For example a 35-9-0-2Ca-2Mg,-3S would indicate a material (fertilizer) with 35% nitrogen, 9% P₂O₅, 0% K₂O, 2% Ca, 2% Mg and 3% S.

When adding any of the NBPT containing solutions described above to aqueous liquid urea fertilizer solutions, additional soluble salts which could be pre-dissolved into an aqueous solution may be added to the solutions to provide other nutrients required for growth of the plant species. Ammonium thiosulfate, ammonium sulfate, potassium chloride, potassium sulfate, manganese (II) sulfate, magnesium sulfate, ammonium phosphate, ammonium polyphosphate, water soluble salts of copper and zinc, ammonium molybdate, sodium molybdate, borates (ammonium, potassium and sodium salts of boric acid) are examples of materials which may be used to provide additional nutrients depending upon the growth requirements of the plant species. In addition to the soluble salts other materials which promote growth such as plant hormones or hormone analogs could be added, chelating agents such as EDTA (ethylene diamine tetraacetic acid), citric acid, gluconic acid, glucoheptonic acid in either the acid form or salt form to prevent metal ion precipitation from the aqueous solution as insoluble hydroxides or carbonates; and materials which cause the liquid to disperse more uniformly across plant leaves such as wetting agents or “spreader stickers” as they are sometimes called in the agricultural industry might be added. Ligninsulfonates which are derived as a byproduct of wood pulping operations are sometimes used in agriculture as both metal sequestering agents and wetting agents could be used. These additional nutrients, growth regulators, chelating agents, sequestering agents or wetting agents could be added singly or in combination.

Denitrification inhibitors are other products which could be added when forming liquid or granular urea products of the present invention, or when treating animal wastes. Examples of denitrification inhibitors include: dicyandiamide (DCD or 2-cyanoguanidine), DMPP (3,4-dimethylpyrazole phosphate), nitrapyrin (2-chloro-6-(trichloromethyl)pyridine). Nitrapyrin is sold as the product N-Serve® by Dow Chemical Company, Michigan USA. To form a liquid product which contains the denitrification inhibitor, the denitrification inhibitor could be added along with one of the NBPT containing solutions of the present invention when preparing the urease inhibited and nitrification protected liquid fertilizer composition. The NBPT solution of the present invention and the denitrification inhibitor could be added as separate ingredients to the solution prepared for field application. If the denitrification inhibitor is soluble in an NBPT containing solution of the present invention, then a product containing both ingredients could be made to supply the urease inhibitor and denitrification inhibitor to prepare urea liquid or granular fertilizers. To make a urease inhibited denitrification inhibited coated granular urea product with other nutrients, the denitrification inhibitor could be added via an aqueous solution used to dilute one of the NBPT containing solutions of the present invention when preparing the coated granular urea product containing additional nutrients. The denitrification inhibitor could be added in a separate coating step.

Other ingredients which could be included in the NBPT containing formulations of the present invention include: odor masking agents such as pine oils, perfumes, etc. Additionally, a second urease inhibitor could be included such as phenylphosphoric diamidate or phenylthiophosphoric diamidate.

### BRIEF DESCRIPTION OF THE DRAWINGS

FIG. 1 is a graph of nitrogen loss for urea ammonium nitrate solution treated with NBPT dissolved in alkyl substituted amino alcohols including mixtures with alkanolamines.

FIG. 2 is a graph of nitrogen loss for urea and urea coated with NBPT dissolved in pH adjusted solutions of alkyl substituted amino alcohols.

FIG. 3 is a graph of cumulative nitrogen loss for urea and urea treated with varying amounts of NBPT dissolved in a pH adjusted crystallization inhibited mixture of TEA, MDEA, and MHEEA.

FIG. 4 is a graph of cumulative nitrogen losses at 7 days and 14 days as a function of the percent NBPT on urea prepared from NBPT dissolved in a pH adjusted crystallization inhibited mixture of TEA, MDEA, MHEEA.

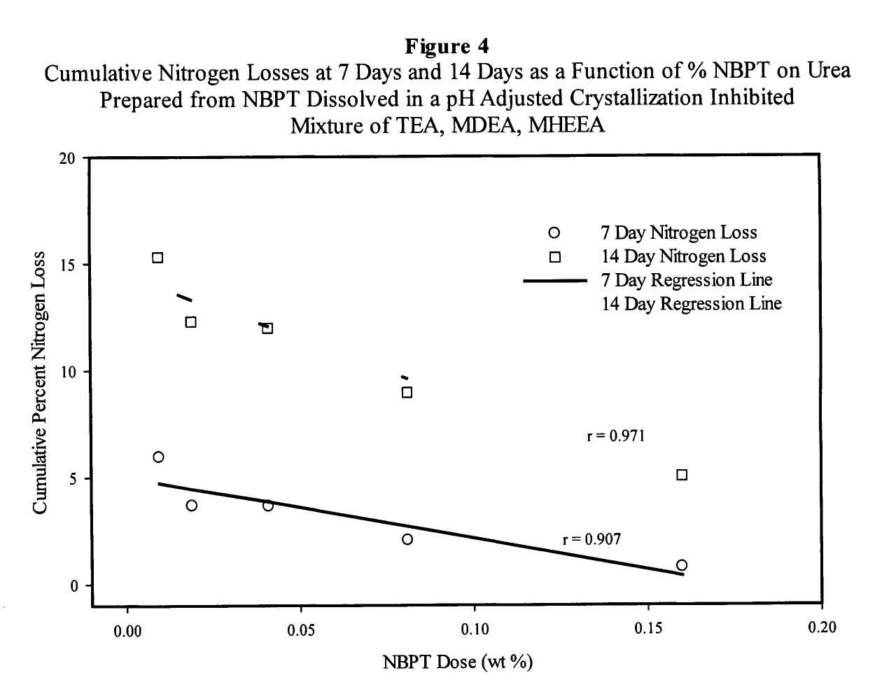

FIG. 5 is a graph of cumulative nitrogen loss for urea and urea coated with NBPT and additional plant nutrients using a single, double and triple layer coating process with pH adjusted solutions of MHEEA, MDEA and TEA and a diluent or binding liquid to supply the liquid to cause the powdered nutrients to adhere.

FIG. 6 is Table 1.

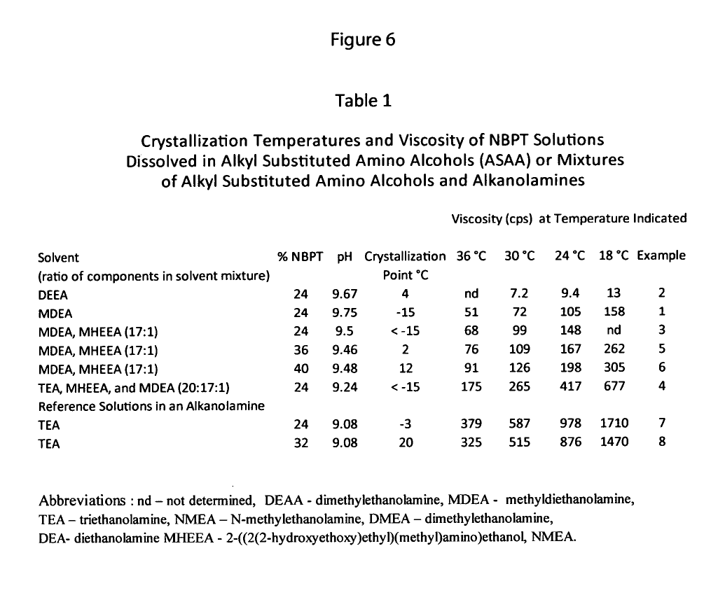

FIG. 7 is Table 2.

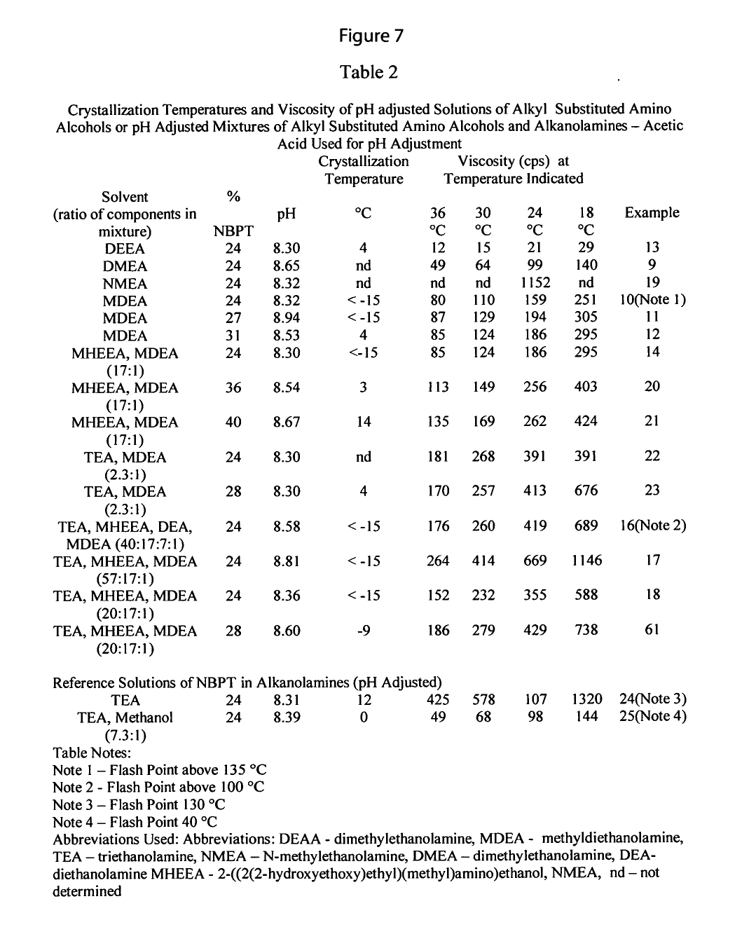

### DETAILED DESCRIPTION OF THE INVENTION

NBPT—N-(n-butylthiophosphoric triamide) is an item of commerce.

Urea ammonium nitrate solutions (UAN) are items of commerce which may contain from 26% N (total) to 32% N (total).

Granular urea is an item of commerce with a fertilizer analysis of 46-0-0. The product may be obtained as granules ranging in size from 1 mm to 10 mm.

The term: alkyl substituted amino alcohol (ASAA) will be used to refer to compounds that could be further classified as N-alkyl or N,N-dialkyl amino alcohols (compounds defined by formula 1 above) and compounds that could be further classified as N-alkyl-N-alkoxy amino alcohols (compounds defined by formula 2 above). It is further understood that the term ASAA may imply a mixture of N-alkyl, N,N-dialkyl, and/or N-alkyl-N-alkoxy amino alcohols.

N-methylethanolamine (NMEA); N-methyldiethanolamine (MDEA), N,N-dimethylethanolamine (DMEA) and N,N-diethylethanolamine (DEEA) are items of commerce which can be used in the practice of the invention. Additionally, mixtures of N-alkyl amino acohols and N-alkyl-N-alkoxy amino alcohols are particularly useful as solvents for NBPT. One such example is a mixture of N-methyldiethanolamine (MDEA) and 2-((2(2-hydroxyethoxy)ethyl)(methyl)amino)ethanol (MHEEA) which is commercially available as the product Amine G2 from Dow Chemical Company, Michigan, USA. The percentages of MDEA and MHEEA present in the mixture actually used will be provided in parenthesis e.g. Amine G2 (5% MDEA, 87% MHEEA).

The term alkanaolamine will be used to refer to compounds which generally could be described as possessing a nitrogen atom which is connected to one or more carbon chains which all possess an alcohol functional group. These compounds are generally the reaction products obtained when oxiranes are reacted with ammonia. The following alkanolamines are items of commerce which could be used in the practice the invention: ethanolamine (MEA), diethanolamine (DEA), triethanolamine (TEA), propanolamine, isopropanolamine (IPA), diisopropanolamine (DIPA).

The term carboxylic acid shall imply a chemical structure containing one or more carboxyl groups. The carbon chain may be from 2-24 carbons in length provided that the material is a liquid. Acetic acid, propionic acid, lactic acid, oleic acid are liquid carboxylic acids which are commercially available and could be used to practice the invention.

To assist in the formulation processes described below, a dye or colorant can be added. Any commonly used colorant may be added to the mixture to provide visual evidence of the uniformity of the distribution of the NBPT containing solutions described below. Depending upon the dye or colorant chosen an alcohol such as methanol, ethanol, propanol, 2-propanol, or butanol e.g. may be required to help disperse the colorant into the NBPT containing solutions described below.

To practice the invention to form an NBPT solution in an alkyl substituted amino alcohol (ASAA), a solution of NBPT is prepared by melting NBPT into liquid ASAA or mixtures of ASAA by heating the liquid mixture containing NBPT to a temperature sufficient to cause the NBPT to melt into the mixture and holding the temperature until all of the NBPT is dissolved. Generally a temperature between 30° C. and 50° C. is adequate to form the initial NBPT solution in the ASAA and preferably a temperature from 40° C. to 45° C. will be used to dissolve the NBPT in the ASAA. Heating may be accomplished by any commonly used heating mechanism such as a jacketed vessel or a heat exchanger system. The initially formed NBPT containing solution in the ASAA may then be modified by adjusting the pH as described below or may be used without pH adjustment to add NBPT to: aqueous urea solutions, granular urea products, and animal wastes as described below.

The NBPT concentration of the NBPT solution in an ASAA amino alcohol or mixture of ASAA will typically lie within the range of 0.5% NBPT to 40% NBPT. The upper limit is determined by the solubility limit of NBPT in the ASAA or mixture of ASAA over the temperature range of use. The lower limit for the NBPT concentration is determined by the application where the solution will be used and will usually be below the solubility limit.

In another embodiment of the invention, the crystallization of NBPT dissolved in an alkanolamine solution or mixture of alkanolamines which might need to be stored down to about −10° C. can be prevented by addition of ASAA or mixture of ASAA. The addition of the ASAA or mixture of ASAA additionally helps to reduce the viscosity of the NBPT solution in an alkanolamine or mixture of alkanolamines. The amount of the ASAA or mixtures of ASAA needed to inhibit the low temperature crystallization of NBPT in alkanolamine solutions or solution in a mixture of alkanolamines will typically range between 15% ASAA and 75% ASAA. The preferred amount will depend upon the NBPT concentration of the final NBPT containing solution being formed and the low temperature requirement for the storage of the NBPT solution.

To form the crystallization protected and viscosity reduced formulations of NBPT in an alkanolamine or mixture of alkanolamines and an ASAA or mixture of ASAA, the alkanolamine and ASAA components may be mixed in any order. The NBPT concentration of the solution formed by combining an ASAA or mixture of ASAA with alkanolamines or a mixture of alkanolamines will typically lie within the range of 0.5% NBPT to 40% NBPT. The upper limit is determined by the solubility limit of NBPT in the solution over the temperature range of use. The lower limit for the NBPT concentration is determined by the application where the solution will be used and will usually be below the solubility limit. The viscosity reduced crystallization inhibited solution of NBPT in a mixture of alkanolamines and ASAA may be used to form pH adjusted mixtures or used to add NBPT to: aqueous urea solutions, granular urea fertilizers, and animal wastes as described below.

When pH control is required for the NBPT solution in an ASAA or mixture of ASAA, the pH of the initial solution of NBPT in an ASAA or mixture of ASAA may be adjusted by reaction of the NBPT containing solution in an ASAA or mixture of ASAA with a liquid carboxylic acid containing from 2 to 24 carbons. The reaction of the ASAA or mixture of ASAA and a carboxylic acid is exothermic and may require cooling. The pH of the carboxylic acid containing mixture should be from 7 to 10, but preferably from 8 to 9. The reaction of an ASAA with a carboxylic acid forms water, the ammonium ion form of the ASAA or a mixture of ammonium ions of ASAA, and the carboxylate ion of the carboxylic acid. When the solution was formed with a mixture of ASAA then a mixture of ammonium ions of the ASAA will be present.

Depending upon the pKₐ of the ASAA or compounds within the mixtures of ASAA, the pKₐ of the carboxylic acid, and the molar ratio of the ASAA or mixture of ASAA to the carboxylic acid, the pH adjusted mixture resulting from the reaction will contain the ASAA, the respective ammonium ions of the ASAA or a mixture ammonium ions of the mixture of ASAA, the ASAA or mixture of ASAA, a carboxylate ion and water. The water will be present in an amount equal to the moles of carboxylic acid that reacted with the ASAA or mixture of ASAA. If the final pH is below the pKₐ of the carboxylic acid and the pKₐ of the ASAA present the pH adjusted mixture will contain a carboxylic acid, carboxylate ion, the ammonium ion of the ASAA and water. Generally, it is preferred that the solution pH be above that of the pKₐ of the carboxylic acid to prevent degradation of NBPT in the acidic solution.

The NBPT concentration in the pH adjusted NBPT solution in an ASAA amino alcohol (or mixture of ASAA) will typically lie within the range of 0.5% NBPT to 40% NBPT. The upper limit is determined by the solubility limit of NBPT in the pH adjusted ASAA solution or the solubility limit of NBPT in the mixture of ASAA over the temperature range of use. The lower limit for the NBPT concentration is determined by the application where the solution will be used and will usually be below the solubility limit.

Generally, the pH adjusted solution of NBPT in an alkyl substituted amino alcohol (or mixture) is required whenever an alkaline solution containing NBPT in an ASAA could cause ammonia to release from an aqueous solution or a solid substrate. Granular urea can contain traces of ammonium carbamate, which when contacted by an alkaline material will form ammonia vapor. Some aqueous urea solutions such as urea ammonium nitrate (UAN) typically have an ammonia odor, thus, the pH adjusted NBPT containing solution does not need to be used.

The pH adjusted NBPT solution in ASAA would be more suited to treatment of manures that typically release ammonia as the urea (from urine) degrades due to bacterial action.

In another embodiment of the invention, ASAA or mixtures of ASAA substituted amino alcohols can be used to prevent NBPT crystallization from solutions prepared from alkanolamines or mixtures of alkanolamines when carboxylic acids have been added. Solutions of alkanolamines can be very unstable when carboxylic acids are present. The crystallization inhibited pH adjusted solutions of NBPT in alkanolamines or mixtures of alkanolamines and ASAA or mixtures of ASAA are formed in the same manner as described above for ASAA.

The NBPT concentration of the pH adjusted NBPT crystallization inhibited solution in a mixture of alkanolamines and ASAA or mixture of ASAA will typically lie within the range of 0.5% NBPT to 40% NBPT. The upper limit is determined by the solubility limit of NBPT in the ASAA over the temperature range of use. The lower limit for the NBPT concentration is determined by the application where the solution will be used and will usually be below the solubility limit.

In the preferred embodiment of the invention for treatment of an aqueous urea solution such as urea ammonium nitrate (UAN solution); the solution of NBPT formed as described above in an ASAA or mixture of ASAA is added to UAN solution to provide an NBPT concentration from 0.005% NBPT to 0.25% NBPT. The amount actually added will depend upon the formulation and would generally reflect the amount of urea within the aqueous urea formulation

Alternatively, the addition of NBPT to an aqueous urea solution such as UAN may be accomplished using a pH adjusted solution of NBPT prepared in an ASAA and or mixture of ASAA. Alternatively, the addition of NBPT to an aqueous urea solution such as UAN may be accomplished using a solution of NBPT prepared in a crystallization inhibited mixture of alkanolamines or mixtures of alkanolamines and an ASAA and or mixture of ASAA. Alternatively, a pH adjusted crystallization inhibited mixture of alkanolamines or mixtures of alkanolamines with ASAA or mixtures of ASAA containing NBPT may be used to treat an aqueous urea solution such as UAN.

When treating an aqueous urea solution with the NBPT containing solutions of the present invention, the NBPT containing solution may be added to a fertilizer applicators tank mix prior to application to the field crop. The term tank mix as used above refers to a solution prepared for application to a field crop. Such solutions are well known in the agricultural industry. The solution could be prepared in bulk at a solution fertilizer distributor for use by the fertilizer applicator.

Fertilizer tank mixes may contain other materials such as additional nutrients, growth promoting compounds such as plant hormones, chelating's agents, wetting agents, and the NBPT dissolved in one of the solutions of the present invention. The mixing order is not critical unless metal salts prone to hydroxide or carbonate formation are included in the tank mix (copper, iron, manganese, zinc e.g.). Whenever a metal salt prone to hydroxide or carbonate formation is included in the tank mix then a chelating agent should be dissolved into the tank mix before adding the metal salt prone to hydroxide or carbonate formation. Examples of chelating agents include, EDTA, HEDTA, citric acid, gluconic acid, glucoheptonic acid and their ammonium, potassium, or sodium salts. Mixtures of chelating agents could be used.

Commercially available chelated metal micronutrient solutions could be used to supply the water soluble metal ions for the tank mix solution containing urea, NBPT and other nutrients.

UAN solutions are sometimes mixed with ammonium thiosulfate solutions to provide both nitrogen and sulfur to plants. In an embodiment of the invention, this N and sulfur supplying UAN-ammonium thiosulfate solution can be treated to protect the urea from urease hydrolysis by adding one of the NBPT solutions of the present invention to the mixture of UAN solution and ammonium thiosulfate solution.

In the preferred embodiment of the invention to form NBPT treated granular urea fertilizers, the pH adjusted NBPT solution in an ASAA or mixture of ASAA would be used. For the treatment of a granular urea fertilizer the concentration of NBPT in the ASAA solution or mixture of ASAA would range from about 0.5% NBPT to about 40% NBPT. After dissolving the NBPT in the ASAA or mixtures of ASAA the carboxylic acid component is added and the pH is adjusted to between 8 and 9. Granular urea is then treated with the solution of NBPT dissolved in the pH adjusted solution of ASAA or mixture of ASAA to achieve an NBPT concentration in the treated urea from 0.005% to 0.25% by weight NBPT. The treatment of the granular urea substrate may be performed in any convenient mixing equipment commonly employed in the fertilizer industry for similar purposes.

The coating of granular urea with NBPT dissolved in a pH adjusted solution of the present invention may be accomplished using any commercially available equipment in which a granular product may be comingled with a liquid. The general procedure is to charge the mixing equipment with granular urea and then add the required amount of the pH adjusted NBPT containing solution of the present invention needed to provide the NBPT for the final product. The combined mass is mixed to distribute the liquid onto the surface of the granular urea particle. The equipment may permit the pH adjusted solution of NBPT to be sprayed onto the granules as they tumble in the mixer or the pH adjusted solution of NBPT may be added into the granules as they tumble within the mixing equipment. The surface wetted granules from addition of the pH adjusted solution containing NBPT are then tumbled until the material has been uniformly distributed across the surface of the granules. The resulting NBPT treated urea product may then be stored or packaged as required. A flow ability aid or desiccant such as gypsum, silica, monoammonium phosphate, potassium sulfate, potassium magnesium sulfate or clay may be required to ensure flow ability of the resulting coated granular product if there is inadequate control of the volume of the pH adjusted NBPT solution. Preferably, the pH adjusted solution containing NBPT is introduced into the mixing equipment via a metering system able to provide reproducible formulations.

The NBPT coated urea product produced with the pH adjusted NBPT containing solution of the present invention may be applied to soil to provide nitrogen needed by plants which contain the valuable urease inhibitor NBPT. The granular product materials containing NBPT may be applied using any routinely used application method such as broadcast by ground or aerial spreading equipment, banding using ground application equipment and spotting techniques wherein the fertilizer is placed next to the plant either above ground or in a depression made into the soil surface next to the plant.

In an alternate embodiment of the invention, the NBPT coated granular urea fertilizer products can be obtained by using a solution of NBPT in an ASAA or mixture of ASAA or a mixture of ASAA. Alternatively, the crystallization inhibited mixture of NBPT in an alkanolamine or mixture of alkanolamines with an ASAA or mixture of ASAA which was not formed by adding carboxylic acids could be used to treat granular urea. The process of forming the NBPT coated granular urea product is the same as described above for the pH adjusted NBPT solutions in ASAA or mixtures of ASAA.

In another embodiment of the invention the treatment of granular urea with NBPT could be performed using a pH adjusted crystallization inhibited solutions of NBPT in alkanolamines and ASAA or mixtures of ASAA. The process of forming the NBPT coated granular urea product is the same as described above for the pH adjusted NBPT solutions in ASAA or mixtures of ASAA.

Other useful granular urea products may be prepared by using the NBPT containing solutions of the present invention as part of the binding agents needed to cause powdered materials to adhere to granular urea. In general there is a liquid to powder ratio which should be determined to cause a powder to adhere to granular urea surface. This liquid to powder ratio depends upon the nature of the powder and will vary with different materials. Generally, the amount of liquid required to form the NBPT coated granular urea products containing additional nutrients is greater than that needed to form a granular urea containing NBPT without additional nutrients; therefore, the NBPT containing solution of the present invention will need to be diluted to avoid potential phytotoxic concentrations of NBPT in the powder coated granular urea product.

In one embodiment of the invention to prepare the NBPT containing granular urea with additional nutrients, the NBPT concentration of the NBPT containing solutions of the present invention may be prepared at a lower concentration. Generally, the concentration of NBPT in a solution of the present invention need to be less than about 5% when preparing NBPT containing granular urea products with additional nutrients.

In another embodiment of the invention the NBPT containing solution in of the present invention may be diluted. The dilution of a NBPT containing solution of the present invention is accomplished by adding other liquids including aqueous mixtures. The resulting diluted NBPT containing mixture derived from the solutions of the present invention can then be used to cause powdered plant nutrient supplying materials to adhere to granular urea.

In another embodiment of the invention, the NBPT concentration of the solutions of the present invention may be reduced at the time the coated products are produced by adding the NBPT containing solution of the present invention along with another solution to the granular urea before adding the powders. This technique is very convenient for batch mixing operations and lab scale production. Any of the diluent solutions described below may be used to provide the additional liquid needed for adhesion of the nutrient powder to the urea.

Examples of solutions which may serve as diluents and binding agents for any of the NBPT containing solutions of the present invention include: UAN solutions, UAN solutions blended with aqueous ammonium thiosulfate solution, aqueous ammonium sulfate solutions, aqueous solutions containing phosphates such as potassium phosphate solutions, ammonium phosphate solutions, ammonium polyphosphate solutions, aqueous solutions of metal nitrates such as nitrates of calcium, copper, iron, magnesium, manganese, potassium, and zinc or mixtures of metal nitrates, or aqueous solutions of metal acetates. Mixtures of the aqueous solutions indicated could be used provided that the materials are chemically compatible with one another. Other examples of solutions which could be used are the binding agents such as Arborite® Binder 77 (aqueous triethanolamine borate) Arborite® Binder 75 (aqueous copper ethanolamine complex and copper (II) borate mixture) both of which are sold by Encee Chemical Sales of North Carolina, USA referenced above. Aqueous carboxylic acid salt solutions not containing NBPT prepared by reacting an ASAA or mixtures of ASAA with or without or alkanolamines and a carboxylic acid may be used as a diluent or binding agent. One example of an aqueous amino alcohol salt solution is an aqueous solution of triethanolamine acetate which may be prepared by reacting acetic acid and triethanolamine. Another example of a binding solution which is an aqueous carboxylic acid salt solution is a solution which contains a mixture of the reaction products of acetic and oleic acid with triethanolamine.

Coated granular urea products containing additional plant nutrients are then prepared from granular urea, a source or sources of the additional nutrients in powdered form and the diluted NBPT containing solution of the present invention described above. Granular urea is first dampened with the diluted NBPT containing solution of the present invention and the materials are mixed to distribute the NBPT containing liquid mixture over the granular urea surface. Any commonly used equipment to comingle a liquid with a granular solid. After distribution of the diluted NBPT containing solution over the granular surface, the additional nutrients in powdered form are added to the dampened mixture and the resulting combined ingredients are further mixed to distribute the powdered materials.

In an alternate embodiment of the invention for forming the powder coated NBPT containing urea fertilizer, an NBPT containing solution of the present invention and a diluent liquid or binding agent are added to the granular urea first, the ingredients are then tumbled, and finally the powdered nutrient source is added and the mixing is continued to distribute the powder throughout the NBPT containing and liquid wetted urea.

In an another alternate embodiment for forming the powder coated NBPT containing urea, powdered materials may be first mixed with the granular urea and then the NBPT containing diluted mixture is sprayed onto a tumbling bed of the dry ingredients to agglomerate the dry materials. This later method is particularly suited to continuous processing.

In an another alternate embodiment for forming the nutrient powder coated NBPT containing coated urea may be prepared from NBPT containing solution of the present invention may be formed without pH adjustment. The process of forming the NBPT coated urea fertilizers with powdered nutrients is the same as that described for using the pH adjusted NBPT solutions of the present invention.

As another embodiment of the present invention to form a nutrient supplying powder coated urea containing NBPT, the granular urea is first treated with the NBPT containing solution and that NBPT coated urea product may be stored until used to form the nutrient powder coated granular urea product. The pre-treatment of the granular urea with the NBPT containing solution has advantages when it is more convenient to separate the NBPT treatment from the nutrient powder coating process, and when it may be desirable to add other external coatings on top of the nutrient powder coating. The central NBPT treated urea granule, thus, is protected from the operations needed to add the additional layers of material to the granular product.

Occasionally, a nutrient powder coated granular urea containing NBPT produced in accordance with the description above may form a pile set when stored. The pile set can be prevented during manufacture or broken after manufacture by adding a metal stearate. Calcium, magnesium and zinc stearates are items of commerce which can be used to disrupt surface adhesions which form in the nutrient powder coated urea containing NBPT. Clays and gypsum will work for the purpose of disrupting the pile set. The pile set disrupting ingredient is typically added after the nutrient powder coated urea with NBPT granule is formed. The pile set disrupting ingredient is typically added in amount which represents less than about 1% of the formulation.

Finally, any of the granular urea products produced as described above may be treated with a de-dusting agent to protect the product from shipping damage. The de-dusting agent may be added at the point of manufacture or the point of shipping. De-dusting agents are common in the fertilizer industry and heavy oils are often used in the fertilizer industry for de-dusting purposes.

As other embodiments of the present invention, animal wastes (manures) may be treated with any of the NBPT containing solutions of the present invention. Preferably pH adjusted solutions would be used. The NBPT solution of the present invention could be sprayed onto the manure before collecting for field application or added to the tank used for field distribution or storage. Once added to the tank it would be necessary to mix the materials to distribute the NBPT throughout the manure. Hays and other bedding materials are often placed in animal stalls and alternatively, the bedding materials could be treated with one of the NBPT containing solutions of the present invention, to decrease the rate of hydrolysis of urea present in the animal urine and reduce ammonia odors. An odor masking compound could be added to the NBPT solution in an ASAA (or mixture) to mask odors from other compounds in the animal wastes.

The NBPT treated manures could be further treated for ecological protection of ground waters by adding a denitrification inhibitor to prevent the conversion of nitrogen materials in the manures to nitrates and subsequently to NO or N₂O, which escape to the atmosphere. These denitrification processes result in the loss of the fertilizer nitrogen value of the applied manure.

Additional volatility control could be achieved if needed by using a second urease inhibitor such as phenylphosphoric diamidate.

The following examples are provided to represent the practice of the invention. Other embodiments could be recognized by anyone skilled in the art by reading of the previous description and examples described below and are properly within the scope of the present invention.

### EXAMPLES

The term powdered when used is understood to refer to any finely divided material with a particle size less than 250 μm (−60 mesh).

In many of the following examples the term melted is used to describe the process of dissolving NBPT into a solvent system. The term melted within this context refers to the heating of the mixture of NBPT and a solvent or mixture of solvent materials to dissolve the NBPT. The dissolution step requires a temperature of between 30° C. and 50° C.

In some of the examples which follow an amine functional group is reacted with a carboxylic acid. The reaction generates an ammonium ion from the amine and a carboxylate ion from the carboxylic acid employed. The reaction, also, generates water in an amount equivalent to the amount of the carboxylic acid which reacted. The quantity of the free amine containing compound remaining was calculated from the moles of acid which reacted with the amine and the initial moles of the amine containing compound present.

In some examples, a mixture of compounds with amine functional groups were used and reacted with a carboxylic acid. In these examples the quantity of free amine remaining was calculated from the moles of acid used and the moles of only one of the amine containing compounds present in the initial mixture. The amine containing compound used for the calculation in this situation is identified as the carboxylate compound from the amine containing group actually used for the calculation in the product composition information of the example.

### ABBREVIATIONS

DEA—diethanolamine

DEEA—N,N-diethylethanolamine

DMEA—N,N-dimethylmonoethanolamine of N,N-dimethylethanolamine

MDEA—N-methyldiethanolamine

NMEA—N-methylmonoethanolamine—N-methyl-ethanolamine

MHEEA—2-((2(2-hydroxyethoxy)ethyl)(methyl)amino)ethanol

NBPT—N-(n-butyl)thiophosphoric triamide

TEA—triethanolamine

UAN—Urea ammonium nitrate (an aqueous urea ammonium nitrate solution)

Amine G2—a mixture of MDEA and MHEEA and other compounds. The formulation used in all examples contained 85% MHEEA, 5% MDEA and 8% other materials. The ratio of MHEEA to MDEA for the Amine G2 mixture used was 17:1.

General Procedures

Viscosity was measured using a Brookfield viscometer (LVDVII). Viscosity was measured at 6° C. temperature intervals from 36° C. to 18° C. (or 12° C.).

Volatile nitrogen losses were measured at 22° C. using equipment described by Woodard et. al. (“Design and Validation of a Laboratory System for Measurement of Volatilized Ammonia” Agronomy Journal Volume 103 Pages 38-44, 2011). Samples were applied to the surface of bare soil with moisture content from 15% to 17% water. The head space was swept at a rate of 1 L/min with moisture saturated air into an acid trap containing 50 mL of 0.04 M H₂SO₄. The ammonia collected by the acid was determined by colorimetric method using a flow injection analyzer (Lachat 8500A).

Examples of NBPT Solutions Prepared from Alkyl Substituted Amino Alcohols

The following examples represent solutions prepared according to the invention containing NBPT dissolved in an alkyl substituted amino alcohols or mixture or mixture of alkyl substituted amino alcohols. The solutions were prepared without pH adjustment. An example of the formation of a crystallization inhibited alkanolamine solution of NBPT prepared using alkyl substituted amino alcohols is included.

Viscosity and crystallization temperatures of the solutions from examples 1-8 are shown in table 1.

### Example 1

A solution containing 24% NBPT was prepared by dissolving 127.2 g of NBPT into 400.0 g of MDEA. The solution was formed by heating the mixture until the temperature reached 45° C. and holding the temperature until the NBPT had dissolved. The solution contained 24.13% NBPT and 75.87% MDEA by weight and had a pH of 9.75.

### Example 2

A solution containing 24% NBPT was prepared by dissolving 127.2 g of NBPT into 400.0 g of DEEA. The solution was as described in example 1. The solution contained 24.13% NBPT and 75.87% DEEA by weight and had a pH of 9.67.

### Example 3

A solution of NBPT was prepared in and mixture of MDEA and MHEEA provided by Amine G2 (5% MDEA, 87% MHEEA). The solution was prepared dissolving 127.2 g of NBPT into 400.0 g of the Amine G2 mixture as described in example 1. The solution contained 24.13% NBPT, 3.79% MDEA and 66.01% MHEEA by weight and had a pH of 9.50. The remaining 6.07% of the solvent mixture was present as unidentified compounds present in the amine G2 mixture.

### Example 4

A crystallization inhibited solution of NBPT was prepared in a mixture of TEA, MDEA and MHEEA. The MDEA and MEA were from the Amine G2 mixture used in example 3. The solution was prepared dissolving 127.20 g of NBPT into 200.00 g of TEA, 200.00 g of Amine G2 (10.00 g of MDEA and 174.00 g of MHEEA) and heating to form the solution as described in example 1. The solution contained: 24.13% NBPT, 37.94% TEA, 1.90% MDEA, 33.00% MHEEA, and 3.03% unidentified compounds present in the Amine G2 mixture by weight. The solution had a pH of 9.24.

### Example 5

A solution of NBPT was prepared in and mixture of MDEA and MHEEA provided by Amine G2 (5% MDEA, 87% MHEEA). The solution was prepared dissolving 157.64 g of NBPT into 280.0 g of the Amine G2 mixture as described in example 1. The solution contained 36.02% NBPT, 3.20% MDEA and 55.66% MHEEA by weight and had a pH of 9.50. The remaining 5.12% of the solvent mixture was present as unidentified compounds present in the amine G2 mixture.

### Example 6

A solution of NBPT was prepared in and mixture of MDEA and MHEEA provided by Amine G2 (5% MDEA, 87% MHEEA). The solution was prepared dissolving 166.75 g of NBPT into 250.00 g of the Amine G2 mixture as described in example 1. The solution contained 40.02% NBPT, 3.00% MDEA and 52.19% MHEEA by weight and had a pH of 9.50. The remaining 4.80% of the solvent mixture was present as unidentified compounds present in the Amine G2 mixture.

### Example 7

A reference solution of NBPT dissolved in triethanolamine was prepared at a concentration of 24% NBPT using 94.80 g of NBPT and 300.00 g of TEA. The solution a pH of 9.08 and contained: 76.00% TEA and 24.00% NBPT.

### Example 8

A reference solution of NBPT dissolved in triethanolamine was prepared at a concentration of 32% NBPT using 117.25 g of NBPT and 250.00 g of TEA. The solution had a pH of 9.08 and contained 67.98% TEA and 32.02% NBPT.

Examples of pH Adjusted NBPT Solutions Prepared with Alkyl Substituted Amino Alcohols

The following examples represent pH adjusted solutions prepared according to the invention containing NBPT dissolved in an alkyl substituted amino alcohols (or mixture of alkyl substituted amino alcohols). Several examples may additionally contain and alkanolamine or mixture of alkanolamines which represent the crystallization inhibited solutions of NBPT in alkanolamines. Acetic acid was commonly used as the acid to adjust the pH although other carboxylic acids would be capable of serving the same purpose.

The reaction of an amine functional group with acetic acid produces ammonium ion of the amine and the acetate ion. To permit calculation of the product composition when two (or more) amine containing groups were present in the solvent mixture; the calculation of the carboxylate ion content was based upon reaction of the acid with only one of the amine containing compounds in the example. The calculation was based usually upon the amine containing compound present in the initial mixture at highest concentration.

Viscosity and crystallization point data for the solutions of example examples 9-14 and 16-25 are shown in table 2.

### Example 9

A pH adjusted solution of NBPT in DMEA was prepared by melting 8.40 g of NBPT into 18.90 g of DMEA at a temperature of 40° C. The pH was then adjusted by adding 6.70 g of glacial acetic acid and then 0.2 g of FDC Yellow#6 was added as a colorant. The final pH was 8.65 and the solution contained: 45.68% of the acetate of DMEA, 24.78% DMEA, 23.39% NBPT, 5.57% H₂O, and 0.57% FDC Yellow#6. All of the acetic acid used in the pH adjustment was reacted with the DMEA present.

### Example 10

A pH adjusted NBPT solution was formed by melting 13.6 g of NBPT into 40.0 g of MDEA at a temperature of 40° C. After dissolving the NBPT 1.45 g of glacial acetic acid was added to adjust the pH and then 0.2 g of FDC Yellow#6 was added. The final solution had a pH of 8.32 and contained: 66.8% MDEA, 24.4% NBPT, and 7.7% of the acetate of MDEA, 0.7% water and 0.04% FDC Yellow#6.

### Example 11

Another pH adjusted solution of NBPT in MDEA was prepared by melting 82.60 g of NBPT into 200.00 g of MDEA at a temperature of 45° C. After the NBPT was dissolved the pH was adjusted to 8.94 with 16.80 g of glacial acetic acid. The final solution contained 54.58% MDEA, 16.54% of the acetate of MDEA, 27.22% NBPT and 1.66% H₂O.

### Example 12

Another pH adjusted solution of NBPT in MDEA was prepared by melting 100.00 g of NBPT into 200.00 g of MDEA at a temperature of 45° C. After the NBPT was dissolved the pH was adjusted to 8.53 with 15.20 g of glacial acetic acid. The final solution contained 53.30% MDEA, 14.01% of the acetate of MDEA, 31.28% NBPT and 1.41% H₂O.

### Example 13

A pH adjusted solution of NBPT dissolved in DEEA was prepared by melting 17.37 g of NBPT into 50.00 g of DEEA and heating to 40° C. to dissolve the NBPT. After dissolving the NBPT the pH was adjusted to 8.30 by adding 4.17 g of glacial acetic acid. The final solution contained: 57.64% DEEA, 16.80% of the acetate of DEA, 23.86% NBPT and 1.70% H₂O.

### Example 14

A pH adjusted solution of NBPT in a mixture of MDEA and MHEEA was prepared by melting 16.40 g of NBPT into 50.00 g of Amine G2 (5% MDEA, 87% MHEEA) at a temperature of 40° C. The pH was then adjusted by adding 1.10 g of glacial acetic acid and then 0.25 g of FDC Yellow#6 was added as a colorant. The final pH was 8.30 and the solution contained: 6.01% of the acetate of MHEEA, 59.50% MHEEA, 3.67% MDEA, 0.48% H₂O, 24.0% NBPT and 0.37% FDC Yellow#6. The final mixture contains 5.88% of unidentified compounds present in the Amine G2 material. For the calculations of the acetate content it was assumed that all of the acetic acid used in the pH adjustment was reacted with the MHEEA present in the Amine G2 material.

### Example 15

A pH adjusted solution of NBPT in a mixture of MDEA and MHEEA (from Amine G2 with 5% MDEA and 87% MHEEA) was formed by melting 34.50 g o NBPT into 100.00 g of Amine G2 and dissolving the NBPT at a temperature of 45° C. 8.60 g of oleic acid was added and the final pH of the mixture was 9.18. The final mixture contained: 57.20% of MHEEA, 9.32% of the oleate of MHEEA, 24.04% NBPT, 3.48% MDEA, 5.58% unidentified compounds (from Amine G2) and 0.38% H₂O. For the calculations of the oleate content it was assumed that all of the oleic acid used in the pH adjustment was reacted with the MHEEA present in the Amine G2 material.

### Example 16

A pH adjusted crystallization inhibited solution of NBPT in mixture of TEA, MHEEA, DEA, and MDEA was prepared by melting 82.4 g of NBPT into a mixture of 150.0 g of commercial TEA (85% TEA, 15% DEA) and 103.5 g Amine G2 (87% MHEEA and 5% MDEA) at a temperature of 40° C. The pH was then adjusted by adding 6.1 g of glacial acetic acid and then 1.1 g of FDC Yellow#6 was added as a colorant. The final pH was 8.58 and the solution contained: 32.6% TEA, 26.1%, 24.0% NBPT, MHEEA, 6.5% DEA, 6.1% TEA acetate, 1.5% MDEA, 0.52% H₂O, and 0.30% FDC Yellow#6. The final product contains 2.4% of unidentified compounds present in the Amine G2. All of the acetic acid used in the pH adjustment was assumed to have reacted with the TEA present in the solvent mixture used to dissolve the NBPT forming TEA acetate.

### Example 17

A pH adjusted crystallization inhibited NBPT containing solution was prepared from a mixture of TEA, MDEA, and MMHEEA by mixing 187.50 g of TEA (99%) with 61.88 g of Amine G2 (87% NHEE, 5% MDEA) and then melting 80.80 g of NBPT at 45° C. into the mixture to dissolve the NBPT. After dissolving the NBPT 2.10 g of glacial acetic acid was added to adjust, then 1.55 g of FDC Yellow#6 was added as a colorant. The final solution had a pH of 8.81 and contained: 52.14% TEA, 4.76% of the acetate of TEA, 15.94% MHEEA, 0.92% MDEA, 23.93% NBPT, 0.38% water, and 1.47% unidentified compounds added with the Amine G2.

### Example 18

Another pH adjusted crystallization inhibited NBPT solution was prepared in a mixture of TEA, MDEA, and MHEEA by adding 100.00 g of TEA (99%) to 100.00 g of Amine G2 (87% MHEEA, 5% MDEA) and then melting NBPT into the solution at 40° C. to dissolve the NBPT. The pH was adjusted to 8.36 by adding 3.60 g of glacial acetic. The final solution contained: 33.81% TEA, 4.67% of the acetate of TEA, 32.32% MHEEA, 24.00% NBPT, 1.86% MDEA, 0.37% H₂O, and 2.97% unidentified materials present in the Amine G2 mixture.

### Example 19

A pH adjusted solution of NBPT dissolved in methylethanolamine (NMEA) was prepared as follows: 112.99 g of NBPT was melted into 200.90 g of NMEA at 45° C. and the pH was adjusted to 8.32 by adding 149.19 g of glacial acetic acid. The final solution contained: 66.00% of the acetate of NMEA, 22.24% NBPT. 2.96% NMEA, and 8.80% H₂O.

### Example 20

A pH adjusted solution of NBPT (36%) in a mixture of MDEA and MHEEA was prepared by melting 161.84 g of NBPT into 280.00 g of Amine G2 (5% MDEA, 87% MHEEA) at a temperature of 40° C. The pH was then adjusted by adding 8.35 g of glacial acetic acid. No colorant was added. The final pH was 8.51 and the solution contained: 6.85% of the acetate of MHEEA, 48.80% MHEEA, 3.10% MDEA, 0.55% H₂O, 35.75 NBPT. The final mixture contains 4.95% of unidentified compounds present in the Amine G2 material. For the calculations of the acetate content it was assumed that all of the acetic acid used in the pH adjustment was reacted with the MHEEA present in the Amine G2 material.

### Example 21

A pH adjusted solution of NBPT (40%) in a mixture of MDEA and MHEEA was prepared by melting 171.25 g of NBPT into 250.00 g of Amine G2 (5% MDEA, 87% MHEEA) at a temperature of 40° C. The pH was then adjusted by adding 7.25 g of glacial acetic acid. No colorant was added. The final pH was 8.67 and the solution contained: 6.27% of the acetate of MHEEA, 45.92% MHEEA, 2.90% MDEA, 0.51% H₂O, 39.76% NBPT, The final mixture contains 4.64% of unidentified compounds present in the Amine G2 material. For the calculations of the acetate content it was assumed that all of the acetic acid used in the pH adjustment was reacted with the MHEEA present in the Amine G2 material.

### Example 22

A pH adjusted solution of NBPT (24%) in a mixture of MDEA and TEA was prepared by melting 123.87 g of NBPT into 93.00 g of MDEA and 217.00 g of TEA at a temperature of 40° C. The pH was then adjusted by adding 6.60 g of glacial acetic acid. The final pH was 8.30 and the solution contained: 5.20% of the acetate of TEA, 45.34% TEA, 21.02% MDEA, 0.45% H₂O, 23.99% NBPT. For the calculations of the acetate content it was assumed that all of the acetic acid used in the pH adjustment was reacted with the TEA.

### Example 23

A pH adjusted solution of NBPT (28%) in a mixture of MDEA and TEA was prepared by melting 121.80 g of NBPT into 150.00 g of MDEA and 150.00 g of TEA at a temperature of 40° C. The pH was then adjusted by adding 11.10 g of glacial acetic acid. The final pH was 8.30 and the solution contained: 8.87.20% of the acetate of TEA, 28.06% TEA, 34.39% MDEA, 0.76% H₂O, 27.92% NBPT. For the calculations of the acetate content it was assumed that all of the acetic acid used in the pH adjustment was reacted with the TEA.

### Example 24

A reference solution of NBPT dissolved in TEA was prepared by using 338.20 g of TEA, 4.75 g of glacial acetic acid and 112.70 g of NBPT. The final solution had a pH of 8.31 and contained: 72.13% TEA, 3.53% of the acetate of TEA, 24.04% NBPT and 0.30% H₂O.

### Example 25

Another reference solution of NBPT dissolved in TEA was prepared by dissolving 64.09 g of NBPT in 170.00 g of TEA and 32.05 g of anhydrous methanol. The pH was adjusted to 8.39 by adding 1.10 g of glacial acetic acid and 0.63 g of FDC Blue #1 and 0.79 g of FDC Red#40 was added. The final solution contained: 62.18% TEA, 11.93% methanol, 1.42% of the acetate of TEA, 23.83% TEA, 0.12% water, 0.23% FDC Blue#1, and 0.29% FDC Red#40.

### Examples of Treating UAN Solutions

Urea ammonium nitrate is a commonly used liquid fertilizer solution containing from 26% N to 32% N. For the examples described below a 32% N solution was used which contains 32.5% urea, 44.5% NH₄NO₃, and 23% H₂O urea and has density of 1.33 g/mL.

In the examples which follow, a weighed quantity of an NBPT containing solution of the present invention was added to 300.00 grams of UAN solution and the combined materials were mixed to distribute the NBPT containing solution throughout the UAN solution.

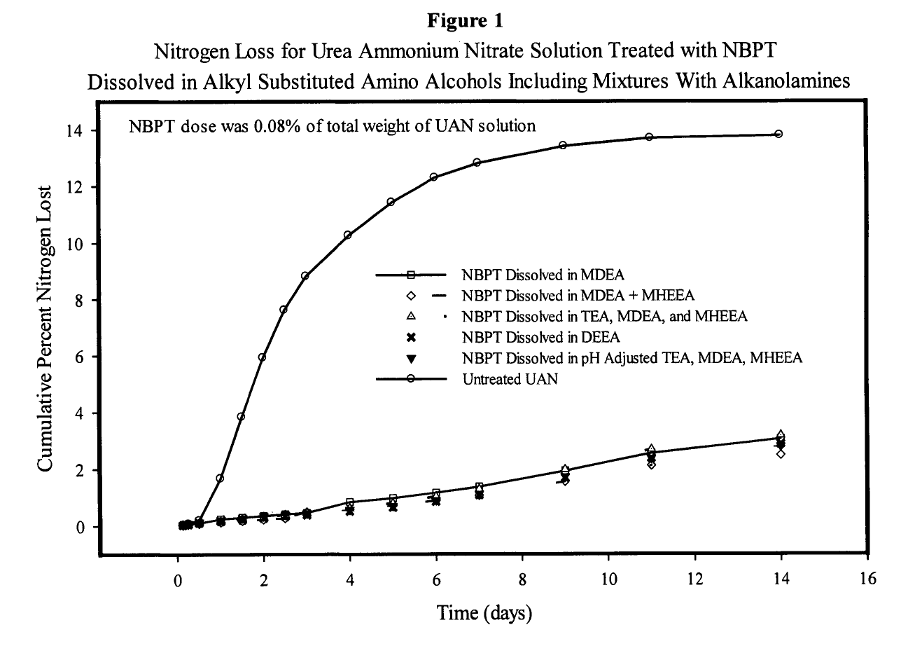

The effect upon volatile nitrogen losses for of the NBPT treatment of the UAN solution is shown in FIG. 1. The volatile nitrogen loss for the treated UAN solutions of examples 26-30 was measured compared to untreated UAN solution urea by applying 275 mcL (μL) of the treated solution to a bare soil surface and collecting the ammonia for a 14 day period.

### Example 26

The NBPT containing solution of example 1 was used to treat UAN solution by adding 1.00 g of the solution of example 1 to 300.00 g of UAN solution. The final treated UAN solution contained 0.080% NBPT on a total weight basis, or 0.23% NBPT on the basis of urea present. The volatile nitrogen loss is shown in FIG. 1.

### Example 27

The NBPT containing solution of example 2 was used to treat UAN solution by adding 1.00 g of the NBPT containing solution of example 2 to 300.00 g of UAN solution. The final treated UAN solution contained 0.080% NBPT on a total weight basis, or 0.23% NBPT on the basis of urea present. The volatile nitrogen loss is shown in FIG. 1.

### Example 28

The solution from example 3 was used to treat UAN solution by adding 1.00 g of the NBPT containing solution of example 3 to 300.00 g of UAN solution. The final mixture contained 0.080% NBPT on a total weight basis, or 0.23% NBPT on the basis of urea present. The volatile nitrogen loss is shown in FIG. 1.

### Example 29

The NBPT containing solution of example 4 was used to treat UAN solution by adding 1.0 g of the NBPT containing solution of example 4 to 300.0 g of UAN solution. The final mixture contained 0.080% NBPT on a total weight basis, or 0.23% NBPT on the basis of urea present. The volatile nitrogen loss is shown in FIG. 1.

### Example 30

The pH adjusted solution of example 18 was used to treat a UAN solution by adding 1.00 g of the pH adjusted NBPT containing solution of example 18 to 300.0 g of UAN solution. The final mixture contained 0.080% NBPT on a total weight basis, or 0.23% NBPT on the basis of urea present. The volatile nitrogen loss is shown in FIG. 1.

### Example 31

An aqueous urea composition with a fertilizer analysis of 28-0-0 with 5S containing 0.025% NBPT (total weight basis) was prepared as follows: to 164 g of a mixture of UAN solution and ammonium thiosulfate solution (described below) 0.17 g of the pH adjusted NBPT containing solution of example 18 was added. The final product contained urea, ammonium thiosulfate, ammonium nitrate and water in the following percentages: 26.23% urea, 35.92% NH₃NO₃, 11.12% ammonium thiosulfate, 26.63% H₂O and 0.10% of the NBPT containing solution of example 18. The NBPT concentration was 0.095% based upon the urea weight (0.025% total weight basis).

The mixture of ammonium thiosulfate solution and UAN solution was formed by adding 71.30 g of ammonium thiosulfate solution (58% ammonium thiosulfate, 42% H₂O) to 300.07 g of UAN (32% N) and mixing to combine the liquids. The final solution had a pH of 8.06 and contained: 35.96% NH₄NO₃, 26.26% urea, 26.64% H₂O and 11.14% ammonium thiosulfate.

### Treating Aqueous Urea Solutions with NBPT Additionally Containing Chelated Micronutrients

In the following examples, the solutions of NBPT in alkyl substituted amino alcohols (or mixtures) were added to an aqueous urea solution and a micronutrient supplying chelated metal solution was added to supply a micronutrient in addition to the nitrogen from urea. The solutions could be used to supply micronutrients to plants or to help plants such as alfalfa, turf grasses, or grasses use for hay to recover from the stress of cutting. The NBPT would protect the urea from breakdown as it was sprayed on the plants to allow uptake of the urea nitrogen.

The solutions of the examples below are formed by adding the chelated metal solution to a premade nitrogen solution containing 20% N (43.4% aqueous urea). The formation of the chelated metal solution used is described in the examples unless a commercially available product was used.

### Example 32

Another aqueous urea solution was formed which contained urea, NBPT and a chelated zinc product. The zinc chelating system included sodium glucoheptonate. The aqueous urea solution containing chelated zinc and NBPT was formed by adding 61.44 g of a zinc glucoheptonate solution (4.38% Zn) described below to 207 g of aqueous 20% N solution (43.4% urea), and then adding 0.30 g of the NBPT containing solution of example 1. The final solution contained 58.6% H₂O, 33.5% urea, 2.9% Zn(NO₃)₂, 0.027% NBPT, ammonium ions and free ammonia added from the zinc glucoheptonate solution described below along with MDEA and its ammonium ion added from the NBPT solution of example 1.

The zinc glucoheptonate solution used in example 33 was formed by dissolving 78.6 g of commercial sodium glucoheptonate crystals into 117.2 g of commercial zinc nitrate solution (17% Zn; 49% Zn(NO₃)₂, 51% H₂O), adding 55.5 g of commercial aqueous ammonia (30% NH₃) and finally 198.70 g of H₂O. The final solution had a pH of 8.00 and contained added ingredients basis): 66.0% H₂O, 17.5% sodium glucoheptonate, 12.8% Zn(NO₃)₂, 3.7% NH₃. The Zn²⁺ ions would react with free ammonia as described in example 1B and the glucoheptonate ion would react with the Zn²⁺ ion to form a complex ion.

### Example 33

An aqueous urea solution was formed which contained urea, NBPT and a chelated iron product. The NBPT containing chelated iron solution in aqueous urea solution was formed by adding 82.1 g of a of ferric ammonium citrate solution described below (3.25% Fe) to 184.00 g of 20% N (43.4% aqueous urea) and then adding 0.27 g of the 24% NBPT containing solution of example 3. The final mixture contained (ingredients basis): 57.8% H₂O, 30.1% urea, 4.33% Fe(NO₃)₃, 0.024% NBPT, and a mixture of the citrate ions, ammonium ion, nitrate ion and free ammonia added from the chelated ferric ammonium citrate solution described below, along with a trace of MDEA, MHEEA and their respective ammonium ions added from the NBPT containing solution of example 3. The fertilizer analysis of the NBPT containing solution of example 3b was 13.6-0-0 with 1% Fe.

The iron ammonium citrate solution (3.25% Fe) used in example 34 was prepared using ferric nitrate solution as follows: 90 gram of commercial citric acid solution (50% citric acid) was added to 118.8 g of commercial aqueous ferric nitrate (10.1% Zn; 43.7 Fe(NO₃)₃, 56.3% H₂O) solution and then 160 g of commercial aqueous ammonia (30% NH₃) was added. The final pH was 8.00 and the final solution contained (as added ingredients): 60.7% H₂O, 12.2% citric acid, 14.1% Fe(NO₃)₃ and 13.0% NH₃. The citric acid was neutralized by the ammonia used so that all citric acid was converted to ammonium citrates and the Fe³⁺ ion would also have reacted with the citrate ions present to form a Fe³⁺ citrate complex ion.

### Example 34

Another aqueous urea solution containing NBPT and chelated iron was prepared. The solution was formed adding 72.8 g of an iron EDTA solution described below to 207.00 g of 20% N solution (43.4% urea) and 0.30 g of the 24% solution of NBPT of example 3. The final solution had a pH of 8.00 and contained (ingredient basis): 55.3% H2O, 32.1% urea, 11.6% Fe(NO3)3 and 0.027% NBPT, and ammonium ions, free ammonia added from the iron EDTA solution described below, along with MDEA and MHEEA and their respective ammonium ions added from the NBPT containing solution of example 3. The fertilizer analysis of the solution was 14.8-0-0 with 1% Fe.

The iron EDTA solution of example 34 was prepared by 54.0 g of EDTA (acid form) to 106.8 g of ferric nitrate solution (17% Fe, 43.7% Fe(NO₃)₃ 56.3% H₂O) and then adding 131 gram of an aqueous 30% ammonia solution. The addition of the ammonia solution caused the acid EDTA to dissolve. The final solution had a pH of 8.00 and contained (ingredient basis): 52.0% H₂O, 18.5% EDTA, 16.0% Fe(NO₃)₃ and 13.5% NH3. The Fe³⁺ present reacted with the EDTA to form an iron EDTA complex

### Example 35

Another aqueous urea solution containing NBPT and chelated iron was formed. The chelating agent for the iron used was a proprietary mixture of iron chelates sold under the name Cee*Quest N5Fe758 by Encee Chemical Sales, referenced above.

The solution was formed as described for example 33 except that 51.8 g of the chelated iron mixture (Cee*Quest N5Fe758) was used along with 164 g of the 20% nitrogen solution and 0.27 g of the NBPT containing solution of 3. The fertilizer analysis of the product was 14.8-0-0 with 1% Fe.

### Examples of Coated Urea Granules Containing NBPT

In the following examples, NBPT coated urea granules were prepared by adding a pH adjusted solution of NBPT dissolved in an alkyl substituted amino alcohol described above to granular urea and then mixing to distribute the liquid over the granular urea surface.

### Example 36

A coated urea granular product was prepared from the pH adjusted NBPT containing solution of example 9 by adding 1.7 g of the NBPT containing solution of example 9 to 500.0 g of granular urea and mixing until the solution was visually distributed throughout the granular material. The final product would have a fertilizer analysis of 45.8-0-0. The product would contain 99.66% urea, 0.080% NBPT, 0.08% DMEA, 0.16% of the acetate of DMEA, 0.02% water. The colorant (FDC Yellow#6) represents 0.002% of the final product.

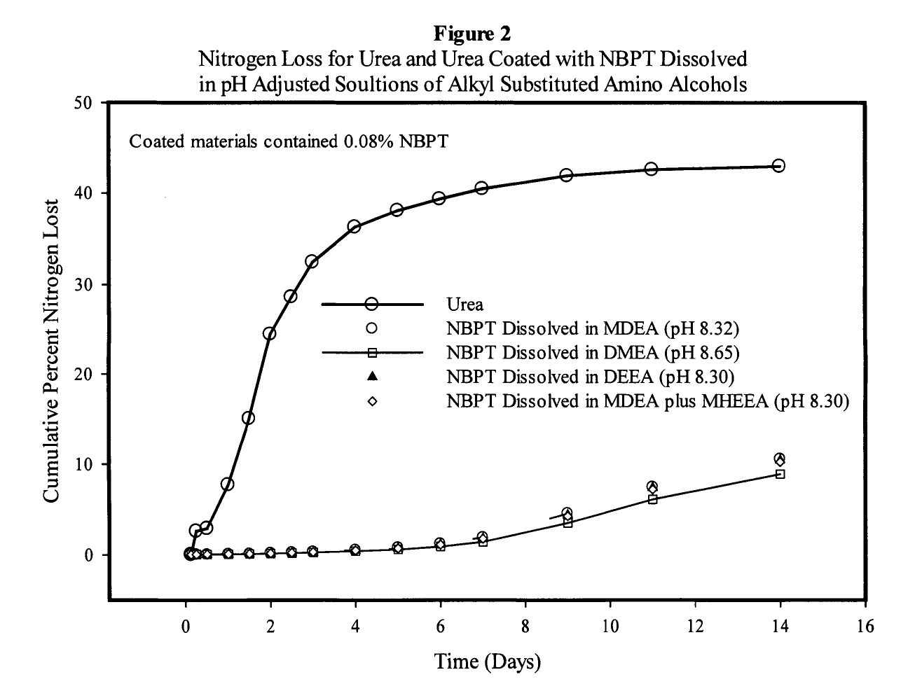

The product of example 36 was tested for volatility control and the results are shown in FIG. 2.

### Example 37

The pH adjusted solution of example 10 was used to coat granular urea by adding 1.7 g of the NBPT containing solution dissolved in MDEA of example 10 to 500.0 g of granular urea and mixing until the solution was visually distributed throughout the granular urea. The coated urea product had a fertilizer analysis of 45.8-0-0 and contained: 99.66% urea, 0.081% NBPT, 0.23% MDEA, 0.03% of the acetate of MDEA. The final product contained 0.002% water and 0.002% of FDC Yellow#6.

The product of example 37 was tested for volatility control and the results are shown in FIG. 2.

### Example 38

A coated urea granular product was prepared from the pH adjusted NBPT containing solution of example 13 dissolved in DEEA by adding 1.7 g of the solution from example 13 to 500.0 g of granular urea and mixing until the solution was visually distributed throughout the granular material. The final product would have a fertilizer analysis of 45.8-0-0. The product would contain 99.66% urea, 0.080% NBPT, 0.21% DEEA, 0.05% of the acetate of DEEA, 0.004% water. The colorant (FDC Yellow#6) represents 0.001% of the final product.

The product of example 38 was tested for volatility control and the results are shown in FIG. 2.

### Example 39

A coated urea granular product was prepared from the pH adjusted NBPT containing solution of example 14 by adding 1.7 g of the solution dissolved in a mixture of MDEA and MHEEA from example 15 to 500.0 g of granular urea and mixing until the solution was visually distributed throughout the granular material. The final product would have a fertilizer analysis of 45.8-0-0. The product would contain 99.66% urea, 0.080% NBPT, 0.21% MHEEA, 0.02% of the acetate of MDEA, 0.01% MDEA, 0.02% undefined compounds present in the Amine G2 used to prepare the solution of example 14, 0.002% water and 0.002% FDC Yellow#6.

The product of example 39 was tested for volatility control and the results are shown in FIG. 2.

### Example 40

A coated urea granular product was prepared from the pH adjusted NBPT containing solution of example 16 by adding 1.7 g of the NBPT containing solution dissolved in a mixture of MDEA, MHEEA, TEA, and DEA of example 16 to 500.0 g of granular urea and mixing until the solution was visually distributed throughout the granular material. The final product would have a fertilizer analysis of 45.8-0-0. The product contained: 99.66% urea, 0.080% NBPT, 0.11% TEA, 0.09% MHEEA, 0.02% DEA, 0.01% TEA acetate, 0.01% MDEA, 0.01% unidentified compounds contributed by the Amine G2 used to prepare the solution of example 16, 0.002% water, and 0.002% colorant (FDC Yellow#6).

### Example 41

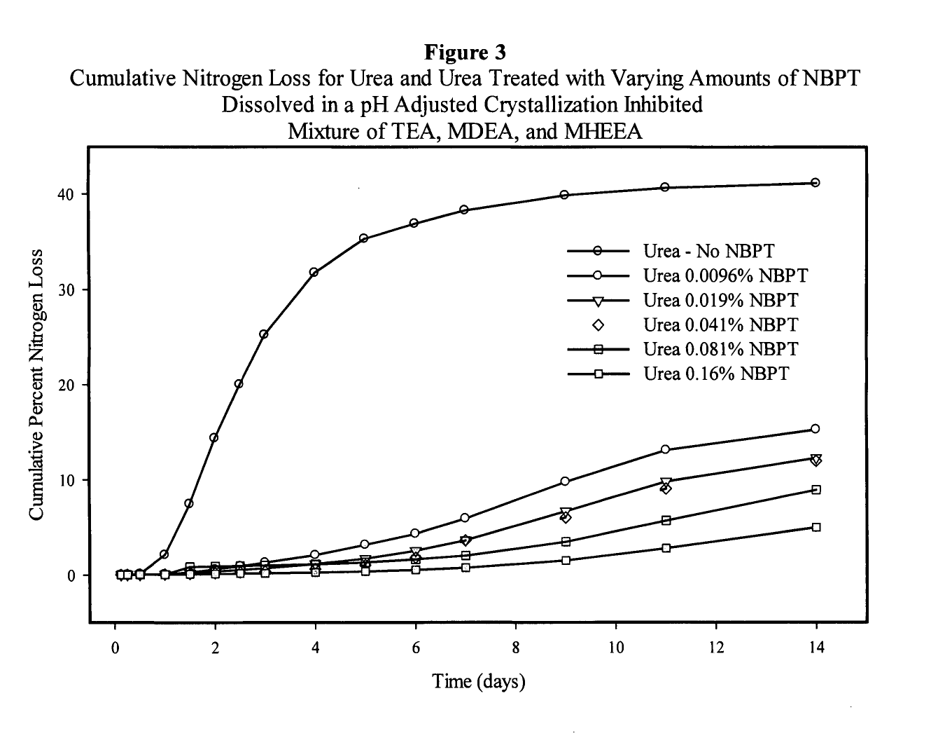

An NBPT coated granular urea was prepared containing 0.01% NBPT was prepared using 0.21 g of the pH adjusted NBPT containing solution of example 17. The final product contained: 99.96% urea; and 0.04% of compounds from the solvent of example 17. The compounds added to the urea granule from the solution of example 17 were: 0.021% TEA, 0.0019% of the acetate of TEA, 0.0064% MHEEA, 0.0004% MDEA, 0.0096% NBPT, 0.0002% water and 0.0006% of unidentified compounds present in the Amine G2 used to prepare the solution of example 17. The volatility control for the product of example 41 is shown in FIG. 3.

### Example 42

A NBPT coated granular urea was prepared containing 0.02% NBPT by using 0.43 g of the pH adjusted NBPT containing solution of example 17. The final product contained: 99.92% urea, and 0.08% of the compounds present in the solution of example 17. The compounds added to the urea granule from the solution of example 17 were: 0.042% TEA, 0.0038% of the acetate of TEA, 0.013% MHEEA, 0.0007% MDEA, 0.019% NBPT, 0.0003% water and 0.0018% of unidentified compounds present in the Amine G2 used to prepare the solution of example 17. The volatility control for the product of example 42 is shown in FIG. 3.

### Example 43

A NBPT coated granular urea product was prepared containing 0.04% NBPT by using 0.86 g of the pH adjusted NBPT containing solution of example 17. The final product contained 99.83% urea, and 0.17% of compounds added from the solution of example 17. The compounds added to the urea granule from the solution of example 17 were: 0.089% TEA, 0.0081% of the acetate of TEA, 0.029% MHEEA, 0.0016% MDEA, 0.041% NBPT, 0.0006% water and 0.0025% of unidentified compounds present in the Amine G2 used to prepare the solution of example 17. The volatility control for the product of example 43 is shown in FIG. 3.

### Example 44

A NBPT coated granular urea was prepared containing 0.08% NBPT by using 1.73 g of the pH adjusted NBPT containing solution of example 17. The final product contained: 99.66% urea, and 0.34% of the compounds present in the solution of example 17. The compounds added to the urea granule from the solution of example 17 were: 0.18% TEA, 0.016% of the acetate of TEA, 0.054% MHEEA, 0.0031% MDEA, 0.081% NBPT, 0.0013% water and 0.005% of unidentified compounds present in the Amine G2 used to prepare the solution of example 17. The volatility control for the product of example 44 is shown in FIG. 3.

### Example 45

A NBPT coated granular urea was prepared containing 0.16% NBPT by using 3.45 g of the pH adjusted NBPT containing solution of example 17. The final product contained: 99.32% urea and 0.68% of the compounds present in the solution of example 17. The compounds added to the urea granule from the solution of example 17 were: 0.35% TEA, 0.032% of the acetate of TEA, 0.11% MHEEA, 0.0063% MDEA, 0.16% NBPT, 0.0026% water and 0.010% of unidentified compounds present in the Amine G2 used to prepare the solution of example 17. The volatility control for the product of example 45 is shown in FIG. 3

### Preparation of Coated Granular Fertilizers Containing NBPT and Additional Nutrients

In the following examples, NBPT containing coated granular urea fertilizers are prepared by addition of a solution of NBPT dissolved in a solution of the present invention described above and adding additional nutrients in in a powdered form of a commercially available plant nutrient supplying substance. The procedure generally followed in the examples involves adding the liquid required to granular urea then mixing to distribute the liquid. After distribution of the liquid, the powder required is added and the combined mass was mixed to coat the liquid wetted urea surface.

The NBPT containing solutions of the present invention were diluted at the time the nutrient powder coated urea product was prepared by first placing the NBPT containing solution onto the granular urea then adding the liquid needed to dilute the NBPT. The diluent liquid is identified in the examples which follow. Other liquid binding agents could be used by someone skilled in the art after confirming their compatibility with the NBPT solutions of the present invention.

### Example 46

A coated granular urea containing 0.08% NBPT with a fertilizer analysis of 40-0-0 with 2.4 Ca, and 1.8 S was prepared by adding 5.49 g of a UAN solution (30% N-32.5% urea, 42% NH₄NO₃, 25.5% H₂O, density 1.302 g/mL) and 1.56 g of the pH adjusted NBPT containing solution dissolved in DMEA of example 9 to 400.00 g of granular urea. The liquid and solid materials were mixed to distribute the liquid over the urea surface and then 52.00 g of gypsum powder (−325 mesh) was added and the combined ingredients were mixed to distribute the powder across the urea surface. The final product contains: 87.13% urea, 11.33% gypsum, 0.16% of the acetate of DMEA, 0.08% DMEA, 0.08% NBPT, 0.02% H₂O, 0.002% FDC Yellow#6 contributed by the solution of example 9 (0.34% of final product) and 0.39% urea, 0.50% NH₄NO₃ and 0.31% water contributed by the UAN solution (30% N solution—1.20% of final product) indicated in the example.

### Example 47

A coated granular urea composition containing 0.08% NBPT with a fertilizer analysis of 37-0-9 with 3S was prepared from the pH adjusted solution of NBPT containing solution of example 10 dissolved in MDEA as follows: 2.04 g of the NBPT containing solution of example 10 and 10.41 g of the triethanolamine acetate solution described below were added to 500.00 g of granular urea. The ingredients were mixed to distribute the liquid throughout the granular urea and then 112.50 g of powdered potassium sulfate (−60 mesh) was added. The combined ingredients were mixed to distribute the powder over the granular urea surface. The final product contained: 80.00% urea, 18.00% K₂SO4, 0.22% MDEA, 0.03% of the acetate of MDEA, 0.08% NBPT, 0.002% H₂O, and 0.0004% of FDC Yellow#6 from the solution of example 10 (0.33% final product) and 0.81% of the acetate of TEA, 0.79% TEA, 0.07% water and 0.003% FDC Yellow#6 from the triethanolamine acetate solution described below (1.67% of final product).

The triethanolamine acetate solution used in example 47 was prepared by adding 68.00 grams of glacial acetic acid to 400.00 g of triethanolamine. 1.00 g of FDC Red #40 was added to color the final solution. The final pH of the mixture was 7.55 and the solution contained: 48.35% triethanolamine acetate, 47.28% triethanolamine, and 4.16% H₂O and 0.20% FDC Yellow#6.

The triethanolamine acetate solution described above is used in several examples which follow for the purpose of providing the liquid needed to properly bind powder(s) to the granular urea surface.

### Example 48

A phosphate coated granular urea fertilizer analysis of 39-9-0 containing 0.06% NBPT was prepared as follows: 1.53 g of the pH adjusted solution of NBPT dissolved in DEEA of example 13 and 12.63 g of the triethanolamine acetate solution of example 47 were added to 500.00 g of granular urea. After distributing the liquid throughout the granular urea surface, 107.31 g of powdered (−80 mesh) monoammonium phosphate (12-52-0) was added and the combined ingredients were mixed to distribute the powder throughout the liquid wetted granular urea. The final product contained: 80.45% urea, 17.27% monoammonium phosphate, 0.14% DEEA, 0.04% of the acetate of DEEA, 0.01% water, and 0.06% NBPT from the solution of example 13 (0.25% of final product), 0.98% of the acetate of TEA, 0.96% TEA, 0.04% water and 0.004% FDC Yellow#6 from the triethanolamine acetate solution of example 47 (2.03% of final product).

### Example 49

A phosphate coated granular urea fertilizer with an analysis of 39-9-0 with 0.12% B containing 0.06% NBPT was prepared as in the example 48, except that 12.63 g of Arborite® Binder 77 (triethanolamine borate 6% B—Encee Chemical Sales, referenced above) replaced the triethanolamine acetate solution. The final product contained: 80.45% urea, 17.27% monoammonium phosphate, 0.14% DEEA, 0.04% of the acetate of DEEA, 0.01% water, and 0.06% NBPT from the solution of example 13 (0.25% of final product), and 2.03% of the boron containing mixture (Arborite® Binder 77).

### Example 50

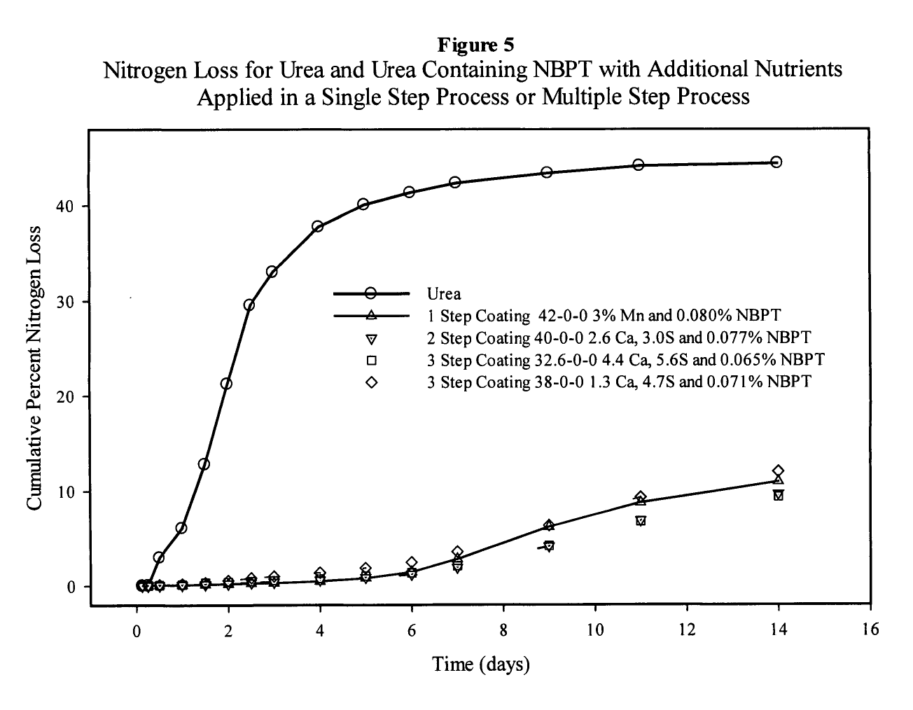

A coated granular urea composition with an analysis of 42-0-0 with 3% Mn containing 0.08% NBPT was prepared as follows: 1.84 g of the pH adjusted solution of NBPT dissolved in a mixture of MHEEA and MDEA of example 14 and 4.95 g of triethanolamine acetate solution of example 47 were added to 500.00 g of granular urea. After distributing the liquid over the urea, 47.15 g of powdered manganese (II) sulfate (35% Mn) was added and the combined ingredients were mixed until the powder was distributed over the urea to coat the urea surface. The final product contained: 90.26% urea, 8.51% manganese (II) sulfate, 0.43% of the acetate of TEA, 0.42% TEA, 0.04% water, and 0.002% FDC Yellow#6 from the triethanolamine acetate solution of example 47 (0.89% of final product), and 0.20% MHEEA, 0.02% of the acetate of MHEEA, 0.01% MDEA, 0.082% NBPT, 0.002% water, 0.001% FDC Yellow#6 and 0.02% unidentified compounds present in the Amine G2 from the solution of example 14 (0.34% of the final product). The fertilizer product of this example developed a pile set upon storage. The volatile nitrogen loss of the final product is shown in FIG. 5.

### Example 51

A gypsum coated urea composition with a fertilizer analysis of 40-0-0 with 1.4 Ca and 1.6 S was prepared as follows: 1.84 g of the pH adjusted NBPT containing solution of example 14 and 8.25 g of UAN solution (30% N see example 46) were added to 500.00 g of granular urea and then 50.00 g of powdered gypsum (−325 mesh) was added. The combined mass was mixed to distribute the gypsum powder. The final product contained: 89.27% urea, 8.93% gypsum, 0.48% urea, 0.62% NH₄NO₃, 0.37% water from the UAN solution (1.47% of final product—from example 46), and 0.19% MHEEA, 0.02% of the acetate of MHEEA, 0.01% MDEA, 0.08% NBPT, 0.001% FDC Yellow#6, 0.02% unidentified compounds present in the Amine G2 used in forming the solution of example 14, and 0.002% water arising from the NBPT solution of example 14 (0.33% of final product).

### Example 52

A zinc coated urea composition was prepared with an analysis of 38-0-0 with 3S and 6 Zn containing 0.08% NBPT was prepared as follows: 2.03 g of the pH adjusted NBPT containing solution dissolved in MDEA and MHEEA of example 14 and 15.55 g of the triethanolamine acetate solution of example 47 were added to 500.00 g of granular urea and then 104.00 g of powdered zinc sulfate monohydrate (17% Zn) was added. The combined mass was mixed to distribute the gypsum powder. The product developed a pile set which was broken by adding 1.95 g of zinc stearate to the material and remixing. The final product contained 80.18% urea, 16.68% zinc sulfate, 0.32% zinc stearate, 1.20% of the acetate of TEA, 1.18% EA, 0.10% water, and 0.01% FDC Yellow#6 from the triethanolamine acetate solution of example 47, and 0.19% MHEEA, 0.02% of the acetate of MHEEA, 0.01% MDEA, 0.08% NBPT, 0.001% FDC Yellow#6, 0.02% unidentified compounds present in the Amine G2 used in forming the solution of example 14, and 0.002% water arising from the NBPT solution of example 14 (0.33% of final product).

### Example 53

A gypsum coated urea composition was prepared with an analysis of 42-0-0 with 1.4 Ca and 1.6S using the pH adjusted NBPT containing solution of example 15 (MHEEA, MDEAA, Oleic Acid) as follows: 1.84 g of the solution of example 15 and 7.00 g of UAN solution (30% N see example 46) were added to 500.00 g of granular urea. After distributing the liquid, 50.00 g of powdered gypsum was added and the combined ingredients were mixed to form the powder coated product. The final product contained: 89.47% urea, 8.95% gypsum, 0.41% urea, 0.45% NH₄NO₃, and 0.39% water from the UAN solution (30% N example 46), and 0.19% MHEEA, 0.03% of the oleate of MHEEA, 0.08% NBPT, 0.01% MDEA, 0.02% unidentified compounds from the Amine G2 used to form the solution of example 15 and 0.001% water from the solution of example 15 (0.33% of final product).

### Example 54

A zinc coated urea composition with a fertilizer analysis of 40-0-0 with 4 Zn and 2S containing 0.08% NBPT was prepared as follows: 1.95 g of the pH adjusted NBPT containing solution of example 18 dissolved in a mixture of TEA, MDEA, MHEEA and 10.00 g of the triethanolamine acetate solution of example 47 were added to 500.00 grams of granular urea and after mixing 65 g of powdered zinc sulfate monohydrate (17% Zn) was added. The combined mass was mixed to distribute the powder across the urea surface and then 5.00 g of zinc stearate was added to inhibit the formation of a pile set as the product was stored. The final product contained: 85.92% urea; 11.17% zinc sulfate; 0.85% zinc stearate; 0.83% of the acetate of TEA, 0.81% TEA, 0.08% water, and 0.003% FDC Yellow #6 from the triethanolamine acetate solution of example 47 (1.72% of final product); and 0.11% TEA, 0.02% of the acetate of TEA, 0.11% MHEEA, 0.08% NBPT, 0.01% MDEA, and 0.01% unidentified compounds from the Amine G2 used in forming the NBPT solution of example 18, and 0.001% water from the NBPT solution of example 18 (0.34% of the final product).

### Example 55

A zinc coated urea composition with a fertilizer analysis of 40-0-0 with 4 Zn and 2S containing 0.08% NBPT was prepared as follows: 1.95 g of the pH adjusted NBPT containing solution of example 18 dissolved in a mixture of TEA, MDEA and MHEEA and 12.35 g UAN solution (30% N) of example 46 were added to 500.00 grams of granular urea and after mixing 65 g of powdered zinc sulfate monohydrate (17% Zn) was added. The combined mass was mixed to distribute the powder across the urea surface and then 3.00 g of zinc stearate was added to inhibit the formation of a pile set as the product was stored. The final product contained: 85.86% urea; 11.16% zinc sulfate; 0.52% zinc stearate; 0.69% urea, 0.89% NH₄NO₃, ands 0.54% water from the UAN solution (30% N-example 46); and 0.11% TEA, 0.02% of the acetate of TEA, 0.11% MHEEA, 0.08% NBPT, 0.01% MDEA, and 0.01% unidentified compounds from the Amine G2 used in forming the NBPT solution of example 18, and 0.001% water from the NBPT solution of example 18 (0.34% of the final product).

### Example 56

A zinc coated urea composition with a fertilizer analysis of 37-0-0 with 6 Zn and 3 S containing 0.08% NBPT was prepared as follows: 2.08 g of the pH adjusted NBPT containing solution of example 18 dissolved in a mixture of TEA, MDEA and MHEEA and 16.50 g UAN solution (30% N) of example 46 were added to 500.00 grams of granular urea and after mixing 104.25 g of powdered zinc sulfate monohydrate (17% Zn) was added. The combined mass was mixed to distribute the powder across the urea surface and then 5.00 g of zinc stearate was added to inhibit the formation of a pile set as the product was stored. The final product contained: 79.64% urea; 16.60% zinc sulfate; 0.80% zinc stearate; 0.85% urea, 1.11% NH4NO3 and 0.67% water from the UAN solution (30% N see example 46); and 0.11% TEA, 0.02% of the acetate of TEA, 0.11% MHEEA, 0.08% NBPT, 0.01% MDEA, and 0.01% unidentified compounds from the Amine G2 used in forming the NBPT solution of example 18, and 0.001% water from the NBPT solution of example 18 (0.33% of final product).

### Example 57

A gypsum coated urea composition with a fertilizer analysis of 40-0-0 with 1.9 Ca and 2.3 S containing 0.08% NBPT was prepared as follows: 1.90 g of the pH adjusted NBPT containing solution of example 18 dissolved in a mixture of TEA, MDEA and MHEEA and 10.22 g aqueous mixture of ammonium thiosulfate with UAN solution (28-0-0 5S) of example 31 were added to 500.00 grams of granular urea and after mixing 73.00 g of powdered gypsum was added. The combined mass was mixed to distribute the powder across the urea surface. The final product contained: 85.45% urea; 12.48% gypsum; 0.46% urea, 0.62% NH₄NO₃, 0.19% ammonium thiosulfate, and 0.48% water from the 28-0-0 5S solution of example 31; and 0.11% TEA, 0.02% of the acetate of TEA, 0.11% MHEEA, 0.08% NBPT, 0.01% MDEA, and 0.01% unidentified compounds from the Amine G2 used in forming the NBPT solution of example 18, and 0.001% water from the NBPT solution of example 18 (0.33% of final product).

### Example 58

A gypsum coated urea granular urea formulation with a fertilizer analysis of 4-0-0 2.6 Ca with 3.0S containing 0.08% NBPT was prepared by a two-step coating process. In the first step, 1.91 g the pH adjusted NBPT solution of example 18 was added to 500.00 g of urea. The materials were mixed to distribute the NBPT containing solution over the urea surface. The NBPT coated material was stored for 24 hours, then 14.56 g of UAN solution (32% N-32.5% urea, 44.5% NH₄NO₃, and 23% H₂O) was added to the NBPT treated urea from step 1 followed by 104.00 g of powdered gypsum. The combined materials were mixed to form the final product. The final product contained: 80.57% urea; 16.76% gypsum; 1.04% NH₄NO₃, 0.76% urea and 0.54% water from the UAN solution (32% N); and 0.11% TEA, 0.02% of the acetate of TEA, 0.11% MHEEA, 0.077% NBPT, 0.01% MDEA, and 0.01% unidentified compounds from the Amine G2 used in forming the NBPT solution of example 18, and 0.001% water from the NBPT solution of example 18 (0.33% of final product). The NBPT content of the final product was 0.077% NBPT. At the end of step 1 the NBPT treated product contained 99.60% urea; 0.14% TEA, 0.019% of the acetate of TEA, 0.13% MHEEA, 0.096% NBPT, 0.0074% MDEA, 0.012% unidentified compounds from the Amine G2 used in forming the NBPT solution of example 18, and 0.0015% water from the NBPT solution of example 18 (0.40% of final product). The product of step 1 contained 0.096% NBPT. The volatile nitrogen loss of the final product is shown in FIG. 5.

### Example 59

A gypsum coated urea granular urea formulation with a fertilizer analysis of 32.6-0-0 4.4 Ca with 5.6S containing 0.065% NBPT was prepared by a three-step coating process. In the first step, 1.91 g of the pH adjusted NBPT solution of example 18 was added to 500.00 g of urea. The materials were mixed to distribute the NBPT containing solution over the urea surface. The NBPT coated material was stored for 72 hours, then 14.56 g of saturated aqueous ammonium sulfate solution (44.44% (NH₄)₂SO₄, 55.56% water) was added to the NBPT treated urea from step 1 followed by 104.00 g of powdered gypsum. The combined materials were mixed to form the product of step 2. The final product was formed by adding 14.56 g of an aqueous saturated ammonium sulfate solution (44.44% (NH₄)₂ SO₄, 55.56% water) to the product of step 2 (620.56 g) and then adding an additional 104.00 g of gypsum. The final product contained: 67.65% urea; 28.14% gypsum; 1.75% ammonium sulfate and 2.19% water from the saturated ammonium sulfate solution; and 0.09% TEA, 0.01% of the acetate of TEA, 0.09% MHEEA, 0.06% NBPT, 0.01% MDEA, 0.01% unidentified compounds from the Amine G2 used in forming the NBPT solution of example 18, and 0.001% water from the NBPT solution of example 18 (0.27% of final product). The NBPT content of the final product was 0.065% NBPT. At the end of step 1 the NBPT treated product contained 99.60% urea; 0.14% TEA, 0.019% of the acetate of TEA, 0.13% MHEEA, 0.096% NBPT, 0.0074% MDEA, 0.012% unidentified compounds from the Amine G2 used in forming the NBPT solution of example 18, and 0.0015% water from the NBPT solution of example 18 (0.40% of final product). The product of step 2 contained 80.58% urea; 16.76% gypsum; 1.05% ammonium sulfate and 1.30% water from the saturated ammonium sulfate solution; 0.10% TEA, 0.01% of the acetate of TEA, 0.10% MHEEA, 0.074% NBPT, 0.0057% MDEA, 0.0091% unidentified compounds from the Amine G2 used in forming the NBPT solution of example 18, and 0.0011% water from the NBPT solution of example 18 (0.31% of final product). The volatile nitrogen loss of the final product is shown in FIG. 5.

### Example 60

Another nitrogen and sulfur sample containing NBPT was prepared with a fertilizer analysis of 38-0-0 1.3 Ca and 4.7 S in a three step coating process. The first coating was prepared using 1.91 g of the NBPT containing solution of example 18 and 500.00 g of urea. The materials were mixed to distribute NBPT over the urea and then 72 hours later the second and third coatings were added. The second coating was formed by adding 10.50 g of saturated ammonium sulfate (44.44% (NH₄)₂SO₄, 55.56% H₂O) to the 501.91 g of product of step 1. 75.00 g of powdered ammonium sulfate was added and the combined materials were mixed to distribute the powder. The third coating was added after the second by adding an additional 7.70 g of the saturated ammonium sulfate solution to the 587.41 g of product from step 2 followed by 55.00 g of gypsum and mixing to distribute the gypsum powder. The final product contained 76.91% urea; 11.54% (NH₄)₂SO₄ (does not include amount from binder); 8.46% gypsum; 1.24% ammonium sulfate and 1.56% water from the saturated ammonium sulfate solution; and 0.10% TEA, 0.01% of the acetate of TEA, 0.10% MHEEA, 0.071% NBPT, 0.006% MDEA, 0.01% unidentified compounds from the Amine G2 used in forming the NBPT solution of example 18, and 0.001% water from the NBPT solution of example 18 (0.29% of final product). The product of step 2 contained: 85.11% urea; 12.77% (NH₄)₂SO₄ (does not include binder); 0.80% ammonium sulfate and 0.99% water from the saturated ammonium sulfate solution; and 0.11% TEA, 0.02% of the acetate of TEA, 0.11% MHEEA, 0.08% NBPT, 0.006% MDEA, 0.01% unidentified compounds from the Amine G2 used in forming the NBPT solution of example 18, and 0.001% water from the NBPT solution of example 18 (0.33% of final product). The product of step 1 contained 99.62% urea and 0.13% TEA, 0.018% of the acetate of TEA, 0.12% MHEEA, 0.091% NBPT, 0.0071% MDEA, 0.011% unidentified compounds from the Amine G2 used in forming the NBPT solution of example 18, and 0.0014% water from the NBPT solution of example 18 (0.38% of final product). The volatile nitrogen loss of the final product is shown in FIG. 5.

### Example 61

An pH adjusted crystallization inhibited NBPT containing solution was prepared in a mixture of TEA, MDEA, MHEEA. The mixture of MDEA and MHEEA was present in the Amine G2 formulation used (87% MHEEA, 5% MDEA). The solution was prepared by melting 119.10 g of NBPT into 150.00 g of TEA and 150 g of the Amine G2 mixture (7.50 g MDEA, 130.50 g MHEEA) at a temperature of 40° C. After dissolving all of the NBPT, 5.40 g of glacial acetic acid was added. The final pH of the solution was 8.60 and the solution contained: 32.04% TEA, 30.68% MHEEA, 27.95% NBPT, 4.41% of the acetate of TEA, 2.82% of unidentified compounds, 1.76% of MDEA and 0.38% water.

Certain modifications and improvements will occur to those skilled in the art upon a reading of the foregoing description. It should be understood that all such modifications and improvements have been deleted herein for the sake of conciseness and readability but are properly within the scope of the following claims.

What is claimed is:

1. A solution for use in reducing nitrogen volatilization comprising N-(n-butyl)-thiophosphoric triamide (NBPT) dissolved in one or more N-alkyl amino alcohols, N,N-dialkyl amino alcohols, N-alkyl-N-alkoxy amino alcohols, and mixtures thereof.

2. The solution of claim 1, wherein said N-alkyl amino alcohols and N,N-dialkyl amino alcohols have the formula: 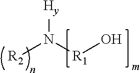
where R₁ is a carbon chain from 2 to 4 carbon atoms and R₂ is a carbon chain with 1 to 4 carbon atoms; m, n, are integers with values of 1 or 2; y is an integer with values of 0 or 1 such that m+n+y=3; and when y=0 then m+n=3. R₁ and/or R₂ may be branched structures in which R₂ does not contain hydroxyl groups.

3. The solution of claim 1, wherein said N-alkyl-N-alkoxy amino alcohols have the formula: 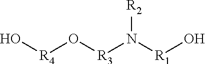
where R₁, R₃ and R₄, are a carbon chains with from 2 to 4 carbon atoms and R₂ may be a carbon chain of 1 to 4 carbon atoms, and R₂ does not contain hydroxyl groups.

4. The solution of claim 1, wherein said N-alkyl amino alcohol is selected from the group consisting of N-methylethanolamine (NMEA), N-ethylethanolamine, N-propylethanolamine, N-isopropylethanolamine, N-butylethanolamine, N-sec-butylethanolamine, N-isobutylethanolamine and N-tert-butylethanolamine, N-methyldiethanolamine (MDEA), N-ethyldiethanolamine, N-propyldiethanolamine, N-isopropyldiethanolamine, N-butyldiethanolamine, N-sec-butyldiethanolamine, N-isobutyldiethanolamine, and N-tert-butyldiethanolamine.

5. The solution of claim 1, wherein said N,N-dialkyl amino alcohol is selected from the group consisting of N,N-dimethylethanolamine (DMEA), N,N-diethylethanolamine (DEEA), N,N-dipropylethanolamine, N,N-diisopropylethanolamine, N,N-dibutylethanolamine, N,N-disecbutylethanolamine, N,N-diisobuytlethanolamine and N—N-tert-butylethanolamine.

6. The solution of claim 1, wherein said N-alkyl-N-alkoxy amino alcohol is selected from the group consisting of 2-((2(2-hydroxyethoxy)ethyl)(methyl)amino)ethanol (MHEEA), 2-(ethyl(2-(2-hydroxyethoxy)ethyl)amino)ethanol, 2-((2-(2-hydroxyethoxy)ethyl)(propyl)amino)ethanol, 3-(ethyl(3-(3-hydroxypropoxy)propyl)amino)propan-1-ol and 3-((3-hydroxypropoxy)propyl)(methyl)amino)propan-1-ol.

7. The solution of claim 1, wherein said solution includes from about 0.5% NBPT to about 40% NBPT.

8. The solution of claim 1, wherein said solution is pH adjusted to a pH of from 7 to 10.

9. The solution of claim 1, further including a co-solvent.

10. The solution of claim 1, further including a denitrification inhibitor.

11. The solution of claim 10, wherein said denitrification inhibitor is selected from the group consisting of dicyandiamide (DCD or 2-cyanoguanidine), DMPP (3,4-dimethylpyrazole phosphate), and nitrapyrin (2-chloro-6-(trichloromethyl)pyridine).

12. The solution of claim 1, wherein said NBPT is dissolved in a mixture of N-methyldiethanolamine (MDEA) and 2-((2(2-hydroxyethoxy)ethyl)(methyl)amino)ethanol (MHEEA).

13. A solution of claim 1, wherein the NBPT is dissolved in a mixture of an N-alkyl, N,N-dialkyl, or N-alkyl-N-alkoxy amino alcohol which additionally contains triethanolamine, diethanolamine and mixtures thereof.

14. A solution of claim 13 in which the pH is adjusted from 7 to 10.

15. A solution of claim 13 wherein the NBP is dissolved in a mixture of N-methyldiethanolamine (MDEA) and 2-((2(2-hydroxyethoxy)ethyl)(methyl)amino)ethanol (MHEEA) and triethanolamine or diethanolamine and mixtures thereof.

16. A method of reducing the nitrogen volatility of a urea fertilizer comprising combining said fertilizer with N-(n-butyl)-thiophosphoric triamide (NBPT) dissolved in one or more N-alkyl amino alcohols, N,N-dialkyl amino alcohols, N-alkyl-N-alkoxy amino alcohols, and mixtures thereof.

17. The method of claim 16, wherein said fertilizer is in aqueous solution.

18. The method of claim 16, wherein said fertilizer is granular and said solution is coated onto said granules.

19. The method of claim 16, wherein said NBPT comprises from about 0.005% to about 0.25% by weight of the combined fertilizer and solution.

20. The method of claim 16, wherein said N-alkyl amino alcohols and N,N-dialkyl amino alcohols have the formula: 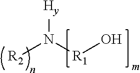
where R₁ is a carbon chain from 2 to 4 carbon atoms and R₂ is a carbon chain with 1 to 4 carbon atoms; m, n, are integers with values of 1 or 2; y is an integer with values of 0 or 1 such that m+n+y=3; and when y=0 then m+n=3. R₁ and/or R₂ may be branched structures in which R₂ does not contain hydroxyl groups.

21. The method of claim 16, wherein said N-alkyl-N-alkoxy amino alcohols have the formula: 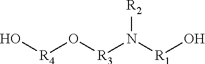
where R₁, R₃ and R₄, are a carbon chains with from 2 to 4 carbon atoms and R₂ may be a carbon chain of 1 to 4 carbon atoms, and R₂ does not contain hydroxyl groups.

22. The method of claim 16, wherein said N-alkyl substituted amino alcohol is selected from the group consisting of N-methylethanolamine (NMEA), N-ethylethanolamine, N-propylethanolamine, N-isopropylethanolamine, N-butylethanolamine, N-sec-butylethanolamine, N-isobutylethanolamine and N-tert-butylethanolamine, N-methyldiethanolamine (MDEA), N-ethyldiethanolamine, N-propyldiethanolamine, N-isopropyldiethanolamine, N-butyldiethanolamine, N-sec-butyldiethanolamine, N-isobutyldiethanolamine, and N-tert-butyldiethanolamine.

23. The method of claim 16, wherein said N,N-dialkyl substituted amino alcohol is selected from the group consisting of N,N-dimethylethanolamine (DMEA), N,N-diethylethanolamine (DEEA), N,N-dipropylethanolamine, N,N-diisopropylethanolamine, N,N-dibutylethanolamine, N,N-disecbutylethanolamine, N,N-diisobuytlethanolamine and N—N-tert-butylethanolamine.

24. The method of claim 16, wherein said N-alkyl-N-alkoxy amino alcohol is selected from the group consisting of 2-((2(2-hydroxyethoxy)ethyl)(methyl)amino)ethanol (MHEEA), 2-(ethyl(2-(2-hydroxyethoxy)ethyl)amino)ethanol, 2-((2-(2-hydroxyethoxy)ethyl)(propyl)amino)ethanol, 3-(ethyl(3-(3-hydroxypropoxy)propyl)amino)propan-1-ol and 3-((3-hydroxypropoxy)propyl)(methyl)amino)propan-1-ol.

25. The method of claim 16, wherein said solution includes from about 0.5% NBPT to about 40% NBPT.

26. The method of claim 16, wherein said solution is pH adjusted to a pH of from 7 to 10.

27. The method of claim 16, further including adding a co-solvent.

28. The method of claim 16, further including adding a denitrification inhibitor.

29. The method of claim 28, wherein said denitrification inhibitor is selected from the group consisting of dicyandiamide (DCD or 2-cyanoguanidine), DMPP (3,4-dimethylpyrazole phosphate), and nitrapyrin (2-chloro-6-(trichloromethyl)pyridine).

30. The method of claim 16, wherein said NBPT is dissolved in a mixture of N-methyldiethanolamine (MDEA) and 2-((2(2-hydroxyethoxy)ethyl)(methyl)amino)ethanol (MHEEA).

31. The method of claim 16, further including adding additional plant nutrients.

32. A method of claim 16, wherein the NBPT is dissolved in a mixture of an N-alkyl, N,N-dialkyl, or N-alkyl-N-alkoxy amino alcohol which additionally contains triethanolamine, diethanolamine and mixtures thereof.

33. A method of claim 32, in which the pH is adjusted from 7 to 10.

34. A method of claim 32 wherein the NBPT is dissolved in a mixture of N-methyldiethanolamine (MDEA) and 2-((2(2-hydroxyethoxy)ethyl)(methyl)amino)ethanol (MHEEA).

35. A urea fertilizer having reduced nitrogen volatility comprised of urea and a solution of N-(n-butyl)-thiophosphoric triamide (NBPT) dissolved in one or more of N-alkyl amino alcohols, N,N-dialkyl amino alcohols, N-alkyl-N-alkoxy amino alcohols, and mixtures thereof.

36. The urea fertilizer of claim 35, wherein said urea fertilizer is in aqueous solution.

37. The urea fertilizer of claim 35, wherein said urea is granular urea, said granular urea being coated with N-(n-butyl)-thiophosphoric triamide (NBPT) dissolved in one or more of N-alkyl amino alcohols, N,N-dialkyl amino alcohols, N-alkyl-N-alkoxy amino alcohols, and mixtures thereof.

38. The urea fertilizer of claim 35, wherein said NBPT comprises from 0.005% to 0.25% of said fertilizer.

39. The urea fertilizer of claim 35, further including additional plant nutrients.

40. The urea fertilizer of claim 35, wherein said solution is less than 5% of said fertilizer.

41. The urea fertilizer of claim 35, further including an aqueous diluent.

42. The urea fertilizer of claim 35, further including a denitrification inhibitor.

43. A method of reducing the odor of animal waste comprising treating said waste with a solution of N-(n-butyl)-thiophosphoric triamide (NBPT) dissolved in one or more N-alkyl amino alcohols, N,N-dialkyl amino alcohols, N-alkyl-N-alkoxy amino alcohols, and mixtures thereof.
